(19) United States
(12) Patent Application Publication
(10) Pub. No.: US 2005/0101983 A1
(43) Pub. Date: May 12, 2005

(54) Method and apparatus for forming apertures in blood vessels

(75) Inventors: Amir Loshakove, Moshav-Bazra (IL); Ido Kilemnik, Herzelia (IL); Gil Hefer, Kfar-Saba (IL)

Correspondence Address: FENSTER & COMPANY INTELLECTUAL PROPERTY 2002 LTD.;C/O REED SMITH LLP, 599 LEXINGTON AVENUE, 29TH FLOOR, NEW YORK, NY, 10022-7650, US

(73) Assignee: By-Pass,Inc., Orangeburg, NY (US)

(21) Appl. No.: 10/668,059

(22) Filed: Sep. 22, 2003

Related U.S. Application Data
(63) Continuation of application No. PCT/IL02/00215, filed on Mar. 18, 2002.
(63) Continuation-in-part of application No. 10/459,373, filed on Jun. 11, 2003.
Application No. 10/459,373 is a continuation of application No. PCT/IL01/01019, filed on Nov. 4, 2001.
(63) Continuation-in-part of application No. 10/402,375, filed on Mar. 27, 2003.
Application No. 10/402,375 is a continuation of application No. PCT/IL01/00903, filed on Sep. 25, 2001.
(63) Continuation-in-part of application No. 10/459,407, filed on Jun. 11, 2003.
Application No. 10/459,407 is a continuation of application No. PCT/IL01/00600, filed on Jun. 28, 2001.
(63) Continuation-in-part of application No. 10/239,387, filed on May 30, 2003.
(63) Continuation-in-part of application No. 10/239,364, filed on May 15, 2003.
(63) Continuation-in-part of application No. 10/239,365, filed on Jun. 2, 2003.
(63) Continuation-in-part of application No. 10/221,982, filed on Sep. 18, 2002.
(63) Continuation-in-part of application No. 10/149,364, filed on Oct. 7, 2002.
(63) Continuation-in-part of application No. 09/936,805, filed on Sep. 17, 2001.
(63) Continuation-in-part of application No. 09/936,806, filed on Sep. 17, 2001.
(63) Continuation-in-part of application No. 09/701,531, filed on Nov. 28, 2000.

(30) Foreign Application Priority Data
May 29, 1998 (IL) 124694
Mar. 19, 1999 (IL) 129067
Jun. 28, 2001 (IL) 144051

(51) Int. Cl. A61B 17/34

(52) U.S. Cl. 606/185

(57) ABSTRACT

Hole-forming apparatus (1900) for forming a hole in a blood vessel, comprising a penetration shaft (1908), having a tip (1904) adapted to be inserted through a wall of a blood vessel; a tissue holder (1986), configured to hold a portion of said wall, said holder (1986) being activated to perform said holding separately from an insertion of said penetration shaft (1908) through said wall; and a cutting surface (1910) adapted to cut through the wall.

### RELATED APPLICATIONS

The present application is related to and is a continuation in part of PCT/IL01/01019 filed on 4 Nov. 2001 (04.11.01), PCT/IL01/00903 filed on 25 Sep. 2001 (25.09.01), U.S. Ser. No. 09/936,806 filed on 17 Sep. 2001 (17.09.01), U.S. Ser. No. 09/936,805 filed on 17 Sep. 2001 (17.09.01), PCT/IL01/00600 filed on 28 Jun. 2001 (28.06.01), PCT/IL01/00267 filed on 20 Mar. 2001 (20.3.01), PCT/IL01/00266 filed on 20 Mar. 2001 (20.03.01), PCT/IL01/00074 filed on 25 Jan. 2001 (25.01.01), PCT/IL01/00069 filed on 24 Jan. 2001 (24.01.01), U.S. Ser. No. 09/701,531 filed on 28 Nov. 2000 (28.11.00), PCT/IL00/00609 filed on 28 Sep. 2000 (28.09.00), and claims the benefit under 119 (e) of U.S. Ser. No. 60/254,689 filed on 11 Dec. 2000 (11.12.00). The disclosures of all of these documents are incorporated herein by reference.

### FIELD OF THE INVENTION

The present invention relates to devices and methods for manipulating blood conduits, for example for forming openings in blood vessels and grafts.

### BACKGROUND OF THE INVENTION

Holes are formed in blood vessels for various reasons, principal among which are (a) for insertion of a tube (and later removing the tube sealing the hole); and (b) forming an anastomosis connection between a graft and the blood vessel.

PCT publication WO 00/74579, the disclosure of which is incorporated herein by reference, describes a hole former in which an outer tube is advanced and optionally rotated to cut into a blood vessel from the outside, while the cut part of the blood vessel is prevented from motion by a barb coupled to the hole former.

U.S. Pat. No. 5,129,913, the disclosure of which is incorporated herein by reference, describes a retracting shearing-cut punch, in which a non-rotating and blunt cutting head is inserted into a slit in a blood vessel and retracted while a base tube having a cutting lip is rotated. This effects a shearing cutting of a portion of the blood vessel as the cutting head is retracted towards and into the base tube.

### SUMMARY OF THE INVENTION

An object of some embodiments of the invention relates to methods for forming holes in blood vessels, using cutting action or other types of action. Other embodiments possibly provide alternative or additional benefits.

Forming a hole in a blood vessel optionally involves three functions: 1) penetrating or transfixing the wall of the blood vessel, 2) holding the wall of the blood vessel, and 3) cutting out a hole in the wall. Holding the wall optionally prevents the cut out plug of tissue from falling into the blood vessel, and/or optionally provides a reference plane to cut against. In some hole formers, two or more of these three (or four) functions are performed by the same element, and one of the functions cannot be performed without also performing one of the other functions. For example, if a penetration shaft used to penetrate the wall has barbs near its tip, it necessarily holds the wall once it has penetrated. Even though the barbs can be pushed through the wall into the blood vessel without engaging the wall immediately, the penetration shaft cannot be pulled back out of the blood vessel, once it has started to penetrate the wall, without having the barbs engage the wall. In this sense, initiating the penetration function makes the holding function inevitable.

An aspect of some embodiments of the invention concerns a hole former in which penetrating the blood vessel wall, at least, is independent of holding the wall and/or a tissue plug. (Cutting the wall may or may not be independent of the other two functions.) For example, a penetration shaft with a sharp tip first penetrates the blood vessel wall, and then a holding shaft with rigid or flexible barbs on its sides enters the blood vessel through the opening made by the penetration shaft, and holds onto the wall by means of the barbs. The barbs may engages the wall from the surface or they may be embedded inside the vessel wall, for example never penetrating to the blood vessel interior. Optionally, the holding shaft is hollow and surrounds the penetration shaft, or the penetration shaft is hollow and surrounds the holding shaft, optionally with slots in the penetration shaft for the barbs to emerge, or the two shafts are side by side.

Alternatively, instead of separate holding and penetration shafts, there are flexible barbs, or other elements for holding the wall, such as an expandable disk, on the sides of the penetration shaft, but they are pressed against the side of the penetration shaft, for example by a hollow outer shaft that surrounds the penetration shaft. When the flexible barbs (or other holding elements) are released, for example by withdrawing the outer shaft, then the barbs come out, and hold the wall of the blood vessel.

In one embodiment of the invention, the tissue holding function is activated by retracting a releaser that is on the side of the penetration shaft or tissue holder. The releaser may, for example compress barbs on the penetration shaft. Alternatively, the barbs are rigid and the releaser hides the barbs, for example, the releaser being resilient or being slotted, or the barb being in a depression in the penetration shaft (or tissue holder).

A potential advantage of having the wall holding function independent of the wall penetration function is that the wall penetration can be reversed, without serious damage to the blood vessel, before the wall is held, if the surgeon finds something wrong with the penetration.

In some embodiments of the invention, the cutting function cannot be performed independently of the penetration function. For example, there is a cutting surface, connected to the penetration shaft in such a way that the cutting surface and penetration shaft move together axially, and the cutting surface cuts out a hole in the blood vessel wall once the penetration shaft has penetrated a given distance into the blood vessel. In other embodiments of the invention, the cutting function cannot be performed independently of the holding function. For example, the cutting surface is connected to the holding shaft. In still other embodiments of the invention, cutting is independent of both penetration and holding.

An aspect of some embodiments of the invention concerns a hole former comprising a penetrating element which penetrates a blood vessel wall, and a protecting element which covers the penetrating element, or otherwise prevents the penetrating element from doing any damage, after the penetrating element has penetrated the wall. In an exemplary embodiment of the present invention it is the protective element which moves or is activated to protect the penetrating element. For example, a penetration shaft with a sharpened tip pierces a blood vessel wall and enters the blood vessel. A hollow protective shaft, surrounding the penetration shaft, then enters the blood vessel through the opening made by the penetration shaft, guided by the penetration shaft, until it covers the tip of the penetration shaft, preventing the sharp tip from damaging the blood vessel wall on the other side. Optionally, the protective shaft also is a holding shaft as described above, and has any of the characteristics described for the holding shaft.

In some embodiments of the invention, the penetration tip has a fixed axial location relative to a cutting edge used to cut the blood vessel wall. The location may be, for example, permanently fixed or fixed once the penetration tip is once advanced, for example using a ratchet mechanism.

An aspect of some embodiments of the invention concerns a hole former which does not penetrate the blood vessel wall in the center of the hole that is later cut, but penetrates the blood vessel asymmetrically, on one side of the hole. A potential advantage of this arrangement is that a wall holding element, also asymmetric, can have an open slot on one side which holds onto the wall, after entering the blood vessel through the opening made by the penetrating element. Optionally, the wall holding element holds onto the wall even after the hole is cut, and even after a tissue plug is removed. With a symmetric hole former, by contrast, the wall holding element generally only holds onto the tissue plug, and the hole former is removed when the hole is cut and the tissue plug is removed.

An aspect of some embodiments of the invention concerns a hole former with an asymmetric blood vessel wall holder, comprising a hollow tube or other member with a slot in one side which receives and holds the blood vessel wall. Optionally the slot includes barbs or other tissue holding elements, for example in a same plane as the tube. Optionally, instead of the wall holder having a complete slot, the sides of the slot are formed by two different parts of the hole former, for example the wall holder and a base. The two parts optionally move relative to each other to adjust the width of the slot, for example to match the thickness of the blood vessel wall, or to adjust the force by which the wall holder holds onto the wall.

An aspect of some embodiments of the invention concerns a hole former with a caliper element which is used to measure the thickness of the blood vessel wall. Optionally, the two sides of the caliper, whose relative position is adjusted while performing the measurement, comprise an adjustable wall-holding element, for example a wall-holding element with an adjustable slot, or barbs whose distance from a base is adjustable. Alternatively or additionally, one side of the caliper is advanced until it touches the inside of the far wall of the blood vessel, while the other side of the caliper touches the outside or the inside of the near wall of the blood vessel, and the thickness of the wall is inferred by subtracting the caliper measurement from a measurement of the outer diameter of the blood vessel. Optionally, instead of or in addition to measuring the thickness of the wall, the force required to change the distance between the two sides of the caliper is used to measure the compressibility of the wall. Knowing the thickness and/or compressibility of the wall may be useful, for example, in order to verify that they are within a range recommended for the medical procedure being performed.

An aspect of some embodiments of the invention concerns a hole former which makes a hole whose boundary is part of an opening made initially by a penetrating element. Optionally, the initial opening is a straight slit, or a slit with less curvature than the rest of the boundary of the hole, and optionally the hole is D-shaped. For example, a penetration head with a sharp tip, located asymmetrically on one side of the hole former, initially makes a straight slit through which to enter the blood vessel. This slit then becomes the straight part of the boundary of a D-shaped hole, when a cutting surface, optionally rotating back and forth as it cuts, makes an arc-shaped cut to complete the hole. A D-shaped hole, particularly in the aorta, may be less subject to tearing at the edges than a circular hole. Alternatively, the initial opening is arc-shaped with the same curvature as the rest of the hole, or with greater curvature, and the hole is circular, or lens-shaped, or another shape.

An aspect of some embodiments of the invention concerns a hole former with a penetration head catch, which couples to a penetration head, when the penetration head has finished penetrating the blood vessel wall. The penetration head catch optionally is attached to a base of the hole former, and keeps the penetration head locked to the base once it has finished penetrating the blood vessel wall. This may prevent the penetration head, which may have a sharp tip, from damaging the blood vessel wall, for example on the opposite side of the blood vessel. Alternatively or additionally, the penetration head catch covers the sharp tip of the penetration head, and may prevent it from damaging the blood vessel wall even without locking the penetration head to the base. Optionally, the penetration head catch is spring loaded and automatically couples to the penetration head when the penetration starts to retract. Alternatively, the penetration head catch is made to couple to the penetration head by the surgeon, for example by a control on the handle of the hole former. Optionally, there is a control on the handle which allows the surgeon to release the penetration head catch after it couples to the penetration head, for example if the penetration head catch was set by mistake.

An aspect of some embodiments of the invention concerns a hole former with a helical penetration shaft and a cutting surface. The penetration shaft turns as it penetrates the blood vessel wall, creating a helical channel in the wall. The penetration shaft can be withdrawn from the wall if desired, at any time, without serious damage to the blood vessel, by turning it in the other direction as it is retracted. If the penetration shaft is retracted without turning it, it will hold onto the wall, and optionally it is used to remove a plug of tissue cut out by the cutting surface after the penetration shaft has penetrated the wall. Optionally, there are two helical penetration shafts with opposite helicity, which penetrate the wall at the same time, to avoid exerting any torque on the blood vessel. Having two penetration shafts with opposite helicity possibly also prevents a cut plug of tissue from falling off the penetration shafts by twisting, as the penetration shafts are retracted without twisting. Optionally, the two helical penetration shafts are located side by side. Alternatively, they are coaxial to each other, or nearly coaxial, but with diameters that are at least slightly different so that they do not interfere with each other. Optionally, if the two helical penetration shafts are side by side, they have the same helicity, which may also prevent a cut plug of tissue from falling off the penetration shafts by twisting.

The diameter of each helix is optionally about as large as possible, while still small enough so that two helixes can fit within the diameter of the cut plug, without interfering with each other. For example, each helix has a diameter of one third or one quarter of the plug diameter. The wire making up each helix optionally has a diameter at least a few times less than the helix diameter, so that the helix can be formed from a straight piece of wire without danger of it cracking, but large enough so that, when the helix is imbedded in the blood vessel wall, it can provide a counter force to the cutting surface, without pulling the helix out of the blood vessel wall, or stretching it past its yield strain. Optionally, the wire is also thick enough so that the helixes will not deform significantly when they penetrate the blood vessel wall. For example, the wire diameter is 20% of the helix diameter, or 10% or 5% of the helix diameter.

In accordance with another aspect of some exemplary embodiments of the invention, a hole former includes a penetration tip which optionally retracts after the tip is inserted through a blood vessel wall, a penetration head that passes through the wall and a base that does not pass through the wall. A cutting lip is provided on the base, to cut the vessel wall. Optionally, the cutting action is assisted by rotation of the base, for example complete and/or oscillatory rotations. Optionally, once some or all of the cutting is completed, the penetration head is retracted relative to the blood vessel, thus removing a plug that is cut out of the vessel. Optionally, the penetration head includes a thickened portion to prevent the plug from slipping off the head. Optionally, the retraction of the penetration head is relative to the base, for example the penetration head being spring loaded. Alternatively or additionally, the retraction is by retraction of the hole former as a whole, possibly advancing an over tube over the base to engage the opening formed in the vessel and prevent leakage.

It should be noted that in some embodiments of the invention, the hole former does not provide any contra. Rather, if any contra is necessary, it is provided by the target vessel itself. The penetration head is provided in these embodiments for preventing the cutting lip from slipping sideways and/or for preventing a cut out plug from falling into the blood vessel.

Optionally, the penetration head has a hollow lumen, which is optionally inner-threaded, barbed or otherwise treated to engage tissue. In an exemplary embodiment of the invention, the lumen is attached to a medicine reservoir inside or outside of the hole former. Alternatively or additionally, the penetration head is threaded on its outside, for example, to assist penetration.

In an alternative embodiment of the invention, cutting lips are provided on the penetration head alternatively or additionally to on the base. Alternatively or additionally to a cutting action, a shearing action is provided by the base and the head sliding by each other. Alternatively or additionally, anvil cutting action is provided by locating tissue between an anvil and a cutting edge. In some, but not all, embodiments, there is relative rotation between the head and the base. In an exemplary embodiment of the invention, the head is retracted towards the base to effect the cutting of a blood vessel from inside of the blood vessel.

Another aspect of some embodiments of the invention relates to protecting an inner leaflet valve of a multi-tool anastomotic delivery system. In an exemplary embodiment of the invention, a same delivery system scaffold is used to deliver a hole former and to deliver an anastomotic connector (or for delivering a different tool). While replacing the two tools a valve is provided in the scaffold to prevent blood leakage from the vessel through the scaffold. In an exemplary embodiment of the invention, the hole former is inserted through the valve while covered while the hole former with a cover (e.g., a silicone tube), to prevent contact between sharp parts of the hole former and the valve. Optionally the cover is designed to be torn off, for example, being perforated and/or includes a rip cord.

An aspect of some embodiments of the invention relates to a hole former comprising a tube having a sharp cutting lip and a lumen in which there is provided means for engaging tissue, for example one way engaging, for example using barbs and/or an inner threading. As the tube is advanced (and/or rotated) against a blood vessel or other tissue, the tissue is cut by the lip and forced into the lumen, where it is engaged. Optionally, the tube comprises an outer threading, for example, to assist advancing into the tissue. Optionally, a central guide, for example a needle, is provided, to stabilize the location of the tube relative to the target tissue. Optionally, the central guide is threaded. The guide may be retractable relative to the tube or not. In different exemplary embodiments, the guide is advanced ahead of the lip, is approximately level with a plane defined by the lip or is retracted from the plane.

An aspect of some embodiments of the invention relates to a retracting hole former, in which the penetration head includes a cutting lip and the head rotates as it is retracted towards a base. Optionally, the base rotates. In an exemplary embodiment of the invention, the cutting lip fits inside the base. Alternatively, the cutting lip fits against the base.

An aspect of some embodiments of the invention relates to a hole former including a receptacle in a distal end of a penetration head for receiving a tissue plug being removed from a vessel wall during the formation of a hole in the vessel wall. Optionally, the receptacle is formed by a cutting lip formed on said penetration head. Alternatively or additionally, a cutting lip is formed on a base portion of said hole former. The cutting lip (one or both, if two) can be of various designs, for example, smooth, serrated and/or oblique. In an exemplary embodiment of the invention, the receptacle is deep enough to contain tissue plugs from one, two or more hole forming activities, even if the plug falls apart.

In an exemplary embodiment of the invention, the receptacle includes a plug extraction means. In one example, a spring element, for example a lump of soft silicon or a metal spring, is provided in the receptacle, so that when the hole forming is completed and the hole former removed from the vessel, the plug is ejected from the hole, at least partly, by the spring element. Alternatively or additionally, an axially retractable catch is provided in the receptacle, which is retracted, for example, manually or by a spring out of said receptacle and/or remains' in place when said penetration head is moved away from said base.

An aspect of some embodiments of the invention relates to a hole former that combines anvil cutting and at least one of knife and shearing cutting for forming a hole in a blood vessel. In an exemplary embodiment of the invention, the anvil cutting is used to cut through an adventitsia of a blood vessel and the other cutting method is used for cutting through an intima of a blood vessel. The different cutting methods may be provided using a same cutting lip or using more than one cutting surface. In one example, an inclined part of the penetration head contacts the base to provide an anvil cutting action, while a cutting lip formed on the penetration head slides past the base to provide knife and/or shearing cutting action in another example, the cutting lip provides knife cutting action until it contacts an inclined portion of the base and provides anvil cutting action.

An aspect of some embodiments of the invention relates to a rotating anvil-cutting hole former. Optionally, at least one of the anvil and the cutting head is spring-loaded so that when the anvil and head meet, one of them can retract, thus preventing and/or reducing damage to the cutting part. In an exemplary embodiment of the invention, the penetration head serves as a cutting part and the base is an anvil and is spring loaded. Optionally, the penetration head is retracted and rotated using a thread. Optionally the head can be rotated an infinite number of times once it reaches the base. Optionally, when the head reaches the base, it slips a thread, allowing the base to spring forward.

An aspect of some embodiments of the invention relates to anvil punching against a resilient material, which may be, for example, on the base or on the penetration head. Optionally, the cutting part of the hole former rotates relative to the anvil part. Optionally, when the penetration head is forcefully retracted, it pushes aside the resilient material and retracts into a predefined axial aperture in the anvil.

An aspect of some embodiments of the invention relates to designing hole former parameters. In an exemplary embodiment of the invention, D designates an outer diameter of a cutting lip, while d designates a minimum diameter of the hole former between the penetration head and the base. In an exemplary embodiment of the invention, the hole remover is designed to achieved a desired hole diameter. Generally, as D is closer to d, the amount of tissue removed by the hole forming operation tend to be smaller, as there is less room for the tissue plug to be contained in during the hole forming operation. While if D is substantially larger than d, a larger hole can be formed, having a diameter approaching and possibly passing D.

An aspect of some embodiments of the invention relates to various designs for a penetration tip and/or a penetration head. In an exemplary embodiment of the invention, the penetration head, which optionally serves as an anvil or as a plug holder for holding the vessel wall, is expandable, for example, as a spiral, as a deformable silicon element or as a plurality of radially extending (and, optionally, interconnected) arms. Alternatively, the penetration head may serve as a cutter, for example, in the spiral embodiment. Optionally, retraction of the penetration tip causes expansion of the penetration head.

Alternatively or additionally, an anvil is provided opposite only some of a circumference of a cutting lip.

In an alternative exemplary embodiment of the invention, the penetration tip and head comprise a threaded tube and the hole forming is performed by retracting the thread relative a base.

In an alternative exemplary embodiment of the invention, a penetration head includes a disk that is inserted on its side and/or in a distorted configuration into the vessel wall after the penetration tip enters the vessel. The disk is then used for the hole forming operation, for example, as an anvil.

In the examples of the threaded head and disk head, the cutting action may be, for example, knife, shearing and/or anvil, optionally utilizing a cutting lip on the penetration head.

In an exemplary embodiment of the invention, the penetration tip has the form of a one, two or more sided knife. Alternatively, the penetration tip has the form of a screw. Alternatively or additionally, the penetration head is deeply scalloped on one, two, three or more sides. Alternatively, the penetration head has a cross-section of a cross or a polygon, rather than having a circular cross-section as in some other embodiments.

In an alternative embodiment of the invention, one, two or more cutting spikes are formed as a cutting lip of the penetration head. The spikes have a wide base and a narrow tip and a cutting surface along their outer edge. In one example, two spikes are provided, with bases that together bridge the entire circumference of the penetration head.

An aspect of some embodiments of the invention relates to a needle-like hole former. In an exemplary embodiment of the invention, the base has the shape of a needle with an aperture, optionally oblique, at its tip. The needle itself may have, for example, a symmetric or an asymmetric conical tip. The edges of the aperture are optionally sharpened. A tissue penetration tip is provided through the aperture and includes a trans-axial extension that has the general profile of the aperture. In use, the penetration tip is inserted into a blood vessel so that the trans-axial extension also passes through the blood vessel wall. The penetration tip is then retracted, pulling the blood vessel towards the base, so that the sharpened lips of the base and/or an optionally sharpened surface of the trans-axial extension cut the vessel wall.

An aspect of some embodiments of the invention relates to marking of punch motion. In an exemplary embodiment of the invention, the hole former includes a visual indication of the relative motion of the penetration head and the base and/or of the base relative to the rest of the hole former. In one example, a slot is formed in the base or an extension of the base, through which a marking on an extension of the penetration head is visible. Optionally, the hole former is provided via a delivery system. In an exemplary embodiment of the invention, the delivery system includes a window for viewing relative motion of the hole former and/or of other delivered tool, such as an anastomotic connector delivery tool, which optionally includes a similar progress indication. Optionally, reaching a desired point of progress is alternatively or additionally marked by a loud mechanical click.

An aspect of some embodiments of the invention relates to a side cutter for a blood vessel. In an exemplary embodiment of the invention, the side cutter includes an L shaped element having a sharpened tip. The tip is poked into a blood vessel and one arm of the L inserted into the blood vessel following the tip. The L element is optionally rotated so that its arm is parallel to the vessel axis. The L element is then retracted relative to a base, providing cutting action by an optional sharpened inner lip on the L and/or shearing action against the base. The base is optionally sharpened. The base may be provided on one sides of the L element or it may sandwich the L element. Optionally, the cutting arm of the L is parallel to the base, alternatively, the arm may be inclined towards the base or away from the base.

There is thus provided in accordance with an exemplary embodiment of the invention, hole forming apparatus for forming a hole in a blood vessel, comprising:
a penetration shaft, having a tip adapted to be inserted through a wall of a blood vessel;
a tissue holder, configured to hold a portion of said wall, said holder being activated to perform said holding separately from an insertion of said penetration shaft through said wall; and
a cutting surface adapted to cut through the wall. Optionally, said tissue holder comprises a rigid barb. Alternatively or additionally, said tissue holder comprises a flexible barb. Alternatively or additionally, said tissue holder comprises a disk.

In an exemplary embodiment of the invention, the tissue holder comprises a hollow tube surrounding the penetration shaft. Optionally, said hollow tube is configured to be advanced along said penetration shaft such that said tip is wholly contained by said hollow tube. Optionally, said hollow tube is long enough to contain said tip at least until said wall is cut through by said cutting surface.

In an exemplary embodiment of the invention, the penetration shaft at least partially encloses said tissue holder. Optionally, said tissue holder comprises at least one flexible tissue holding element and wherein the penetration shaft comprises at least one slot wide enough to receive said flexible element therethrough and wherein said tissue holder and said penetration shaft are configured for selectively positioning said penetration shaft relative to said tissue holder such that said tissue holding element and said slot align. Optionally, said tissue holder and said penetration shaft are configured such that said tissue holding element is axially displaced from said slot. Alternatively or additionally, said tissue holder and said penetration shaft are configured such that said tissue holding element is angularly displaced from said slot.

In an exemplary embodiment of the invention, said tissue holder lies alongside said penetration shaft.

In an exemplary embodiment of the invention, said tissue holder is mounted on said penetration shaft and comprising a holder releaser configured to selectively release said holder to hold tissue. Optionally, said holder releaser comprises a hollow shaft which at least partially encloses said penetration shaft. Alternatively, the holder releaser lies alongside said penetration shaft.

In an exemplary embodiment of the invention, said holder releaser covers said tissue holder during said insertion of said penetration shaft into said wall. Optionally, said holder releaser resiliently compresses said tissue holder during said insertion of said penetration shaft into said wall.

In an exemplary embodiment of the invention, said tissue holder is configured to be advanced along said penetration shaft after said insertion. Alternatively or additionally, said tissue holder is configured to be axially moved relative to said penetration shaft, thereby activating said tissue holder.

In an exemplary embodiment of the invention, said tissue holder is configured to be rotated relative to said penetration shaft, thereby activating said tissue holder.

In an exemplary embodiment of the invention, said tissue holder is configured to retract relative to said cutting surface during said hole forming. Optionally, said tissue holder is spring loaded to retract. Alternatively or additionally, said retraction is mechanically coupled to a rotation of said cutting surface.

In an exemplary embodiment of the invention, said penetration shaft has a fixed axial position relative to said cutting surface at least after said insertion.

In an exemplary embodiment of the invention, said penetration shaft is axially retractable relative to said cutting surface.

In an exemplary embodiment of the invention, said cutting surface is configured to cut by rotation. Optionally, said cutting surface is not rotationally fixed to said tissue holder.

In an exemplary embodiment of the invention, said cutting surface is configured to cut by from an opposite side of said wall from said penetration tip.

In an exemplary embodiment of the invention, said cutting surface is configured to cut by from a same side of said wall from said penetration tip.

In an exemplary embodiment of the invention, said penetration tip is configured to enter said wall from an outside of said vessel.

In an exemplary embodiment of the invention, said tissue holder engages a wall of said vessel.

In an exemplary embodiment of the invention, said tissue holder contacts a wall of said vessel at a stop location and thereby prevents relative motion of said wall in a direction of said stop location.

There is also provided in accordance with an exemplary embodiment of the invention, hole forming apparatus for forming an opening in a blood vessel, comprising:
a penetration shaft, having a tip adapted to be inserted through a wall of a blood vessel;
a cutting surface adapted to cut through the wall and having a fixed axial position relative to said penetration shaft tip; and
a tip protector, axially movable to protect the tip of the penetration shaft from damaging the blood vessel after the penetration shaft is inserted through the wall of the blood vessel. Optionally, said tip protector comprises at least one tissue holding element configured to hold at least a portion of said wall after said insertion. Optionally, said tip protector has a length, distal of said tissue holder, greater than an axial distance between said tip and said cutting surface.

In an exemplary embodiment of the invention, the tip protector comprises a hollow tube which surrounds the penetration shaft.

There is also provide din accordance with an exemplary embodiment of the invention, hole forming apparatus for forming a hole in a blood vessel, comprising:
a slit-forming penetration head having a tip on a distal end thereof and configured to be inserted into, and form a slit, in a blood vessel wall; and
a cutting surface not-contiguous with said tip, configured to cut said wall such that said cut and said slit link to provide a boundary cut of said wall, defining said hole. Optionally, said cutting surface is formed on a proximal side of said head. Alternatively or additionally, said tip is asymmetrically located on said head relative to an axis of said apparatus.

In an exemplary embodiment of the invention, the penetration head is solid.

Alternatively, the penetration head is hollow. Optionally, the apparatus includes at least one head tissue holder positioned inside said head and configured to prevent a cut out portion of the wall of the blood vessel from passing through said head in a direction of said tip. Optionally, said tissue holder is fixed to said head. Alternatively or additionally, said tissue holder comprises a hook.

In an exemplary embodiment of the invention, said penetration head has an arcuate profile when viewed along its axis.

In an exemplary embodiment of the invention, the apparatus includes a sharpened tip at a proximal end of the penetration head. Optionally, said sharpened tip is on a same plane as said penetration tip and an axis of said penetration head.

In an exemplary embodiment of the invention, the apparatus includes an anchor comprising a member defining a trans-axial slot, said slot having a width sufficient to receive a thickness of said wall. Optionally, said member comprises a tube defining a lumen having a diameter sufficient to enclose said penetration head. Alternatively, said member only partly surrounds said penetration head.

In an exemplary embodiment of the invention, the apparatus includes an anchor tissue holder attached to said anchor, which anchor tissue holder prevents a cut out portion of the wall of the blood vessel from passing into the blood vessel.

In an exemplary embodiment of the invention, said member has a distal end. Optionally, said distal end comprises an anchor cutting surface. Alternatively, said distal end comprises said cutting surface.

In an exemplary embodiment of the invention, said distal end and a proximal end of said penetration head cooperate to provide a scissors cutting action. Alternatively, said distal end and a proximal end of said penetration head cooperate to provide an anvil cutting action.

There is also provided in accordance with an exemplary embodiment of the invention, hole forming apparatus for forming a hole in a blood vessel, comprising:
a penetration head adapted to penetrate a blood vessel wall;
a slotted anchor, defining a trans-axial slot having a width sufficient to receive a thickness of said wall; and
a cutting surface configured to cut said wall while said wall is held by said slotted anchor. Optionally, said anchor comprises a trans-axially-slotted tube. Alternatively or additionally, the penetration head comprises a distal portion of the anchor. Alternatively or additionally, the apparatus includes a tissue holder which prevents a cut out portion of the wall of the blood vessel from passing into the blood vessel.

In an exemplary embodiment of the invention, said cutting surface is formed on a proximal side of said penetration head.

There is also provided in accordance with an exemplary embodiment of the invention, hole forming apparatus for forming a hole in a blood vessel, comprising:
a penetration head having a tip adapted to penetrate a blood vessel wall and defining an axial lumen; and
a tip protector configured to pass through said lumen and protect said tip from damaging the blood vessel after said penetration. Optionally, said tip protector comprises a folded tab defining a receptacle configured to receive said tip therein. Optionally, said tab is resiliently distorted during passage through said lumen.

In an exemplary embodiment of the invention, said tip protector has a fixed axial location relative to said apparatus such that retraction of said penetration head causes said tip protector to pass through said lumen and protect said tip. Alternatively or additionally, said tip protector comprises a control for manually positioning said tip protector to selectively protect said tip.

There is also provided in accordance with an exemplary embodiment of the invention, hole forming apparatus for forming a hole in a blood vessel, comprising:
a tissue holder configured to be inserted through a blood vessel wall;
a base configured to be positioning on an opposite side of said wall; and
a display coupled to said tissue holder and said base and configured to show an indication based on a relative distance between said tissue holder and said base. Optionally, said tissue holder includes at least one tissue retraction prevention element which prevents retraction of said tissue holder back through said wall. Alternatively or additionally, said display is mechanically coupled to said holder and said base. Alternatively or additionally, said base contacts said wall with a blood vessel wall cutting surface. Alternatively or additionally, the apparatus includes a spring which retracts said holder relative to said base. Alternatively or additionally, said display converts said distance into a measure of compressibility of said wall. Alternatively or additionally, said display converts said distance into a measure of a thickness of said wall.

There is also provided in accordance with an exemplary embodiment of the invention, hole forming apparatus for forming a hole in a blood vessel, comprising:
a penetration head mounted on a shaft and adapted to penetrate a blood vessel wall; and
a tissue receptacle configured to receive said wall after said penetration,
wherein said shaft is not co-axial with said tissue receptacle. Optionally, said shaft is outside of said tissue receptacle.

There is also provided in accordance with an exemplary embodiment of the invention, hole forming apparatus for forming a hole in a blood vessel, comprising:
a plurality of helical coils adapted to penetrate and engage a blood vessel wall; and
a base defining a cutting surface for cutting said wall and configured to be axially moved relative to said helical coils. Optionally, said coils include at least two coils having opposite helicity. Alternatively or additionally, said coils include at least two coaxial coils. Alternatively or additionally, said coils include at least two non-coaxial coils.

There is also provided in accordance with an exemplary embodiment of the invention, a method of forming a hole in the wall of a vessel, comprising:
providing a hole former comprising a shaft having a penetrating tip formed at a distal end of the shaft, an outer shaft disposed about the shaft and configured to be slideable with respect to the shaft, the outer shaft having at least one projecting element, and a base disposed about the outer shaft having a cutting lip, the base configured to be slideable relative to the outer shaft;
penetrating a vessel wall with the penetrating tip to form an opening;
passing the outer shaft and the at least one projecting element through the opening; and
cutting the vessel wall with the cutting lip of the base to form a hole in the wall of the vessel. Optionally, the method comprises retracting the outer shaft such that the at least one projecting element contacts the inner surface of the vessel wall prior to said cutting. Alternatively or additionally, the shaft and the base are fixed relative to one another. Alternatively, the shaft is moveable independently of the base. Optionally, the penetrating tip is retracted into the outer shaft one the outer shaft has passed through the opening.

In an exemplary embodiment of the invention, the outer shaft shields the inner surface of the vessel wall from the penetrating tip during the cutting step.

In an exemplary embodiment of the invention, the base is rotated to cut the hole in the vessel wall.

### BRIEF DESCRIPTION OF THE FIGURES

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements or parts which appear in more than one figure are preferably labeled with a same or similar number in all the figures in which they appear, in which:

FIG. 1A illustrates a hole former having an outer cutting lip, in accordance with an exemplary embodiment of the invention;

FIG. 1B illustrates a hole former having an inner cutting lip, in accordance with an exemplary embodiment of the invention;

FIGS. 2A-2E are cut-through views of an exemplary hole former, in accordance with an exemplary embodiment of the invention;

FIG. 3 illustrates various dimensions of a penetration head that may be relevant in accordance with an exemplary embodiment of the invention;

FIGS. 4A and 4B illustrate plug removal mechanisms in accordance with an exemplary embodiment of the invention;

FIG. 5 illustrates a base retraction mechanism, in accordance with an exemplary embodiment of the invention;

FIG. 6 illustrates an alternative hole former, in accordance with an exemplary embodiment of the invention;

FIGS. 7A-7I illustrate various penetration tip and penetration head designs, in accordance with exemplary embodiments of the invention;

FIGS. 8A and 8B illustrate an expanding penetration head, in accordance with an exemplary embodiment of the invention;

FIGS. 9A and 9B illustrate an alternative expanding penetration head, in accordance with an exemplary embodiment of the invention;

FIGS. 10A and 10B illustrate another alternative expanding penetration head, in accordance with an exemplary embodiment of the invention;

FIGS. 11A and 11B illustrate a geometry changing anvil, in accordance with an exemplary embodiment of the invention;

FIG. 12 illustrates a resilient anvil hole former, in accordance with an exemplary embodiment of the invention;

FIG. 13 illustrates a thread-type penetration head, in accordance with an exemplary embodiment of the invention;

FIGS. 14A and 14B illustrate a needle-type hole former, in accordance with an exemplary embodiment of the invention;

FIGS. 15A and 15B illustrate two variants of an incision maker, in accordance with an exemplary embodiment of the invention;

FIG. 16A and 16B illustrate a hole former in accordance with an alternative embodiment of the invention;

FIGS. 17A-17E illustrate the use of the hole former of FIG. 16, in accordance with an exemplary embodiment of the invention;

FIG. 18 illustrates a tip of a hole former in accordance with an alternative embodiment of the invention;

FIG. 19A is a perspective cut-away side view, and FIG. 19B is a cross-sectional side view, of a hole former, according to another exemplary embodiment of the invention;

FIGS. 19C, 19D, 19E, 19F, and 19G are cross-sectional side views showing different stages in the process of a hole former making a hole in a blood vessel, using the device of FIGS. 19A and 19B;

FIG. 20A is a see-through side view of a hole former according to another exemplary embodiment of the invention;

FIGS. 20B, 20C, 20D, and 20E are a series of four side views of the device of FIG. 20A, showing how the penetration tip moves with respect to the rest of the hole former;

FIG. 20F is an axial view of the cuts made in a blood vessel by the device of FIG. 20A;

FIG. 20G is a perspective view of the device of FIG. 20A;

FIGS. 20H and 20I are side cross-sectional views of the device of FIG. 20A and a blood vessel wall, showing two steps in the process of cutting a hole in the wall;

FIGS. 21A and 21B are side cross-sectional views of a hole former according to an exemplary embodiment of the invention; and

FIGS. 22A-22E show a hole former with two helical penetration shafts, in accordance with an exemplary embodiment of the invention.

### DETAILED DESCRIPTION OF EXEMPLARY EMBODIMENTS

Side to end anastomosis connections typically require an opening to be made in the “side” vessel, which is typically a target vessel. If an incision is made in the side vessel, expanding the incision to an elliptical or circular opening, as typically required in an anastomosis connection, may cause tearing and/or distortion of the target vessel. An alternative method is to punch or cut out a hole in the vessel (e.g., using the methods described in the background). However, the inventors have found that such punching may create a hole with one or more tears on its circumference. For example, punching a 2.5 mm diameter hole in an aorta, typically causes a tear, which, once the anastomosis is completed, may expand and cause a leak. In some cases, the size of the hole in the aorta has been shown to affect the probability of causing a tear, however, a minimal hole size may be required in order to prevent distortion of the aorta when performing an anastomosis of a larger diameter.

A blood vessel is formed of several layers. The outermost layer is a tough fibrous layer called the adventitsia. The innermost layer is called the intima. The inventors have found that if the cutting proceeds from the outside in, the adventitsia may catch on the cutting element and distort the intima before it is cut. In addition, the inventors have determined that different cutting methods may be useful for the different layers of the blood vessel.

Once a portion (a plug) is cut out of the vessel wall, it is typically desirable to prevent the plug from falling into the blood flow. In addition, the plug may fall apart during or after the hole formation.

One or more of the above problems is solved by some of the embodiments of the invention.

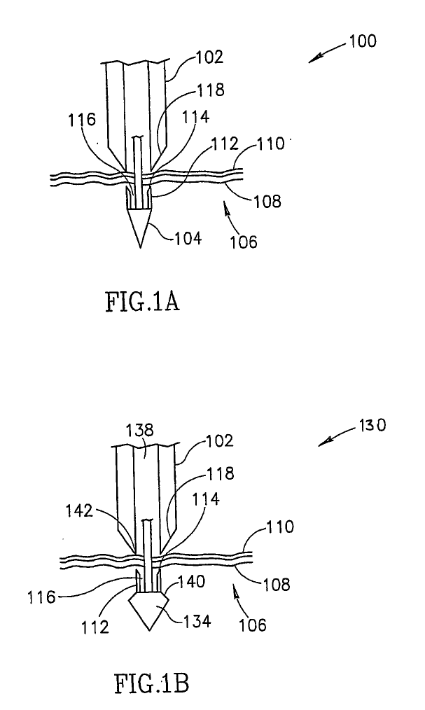

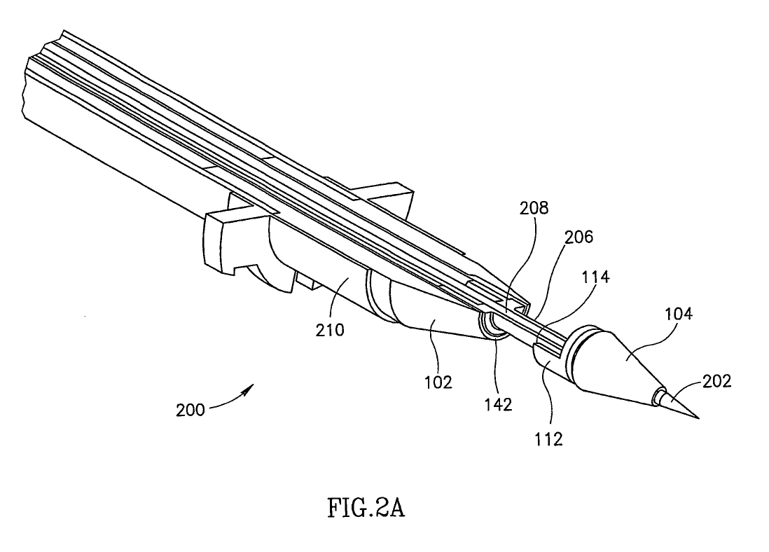

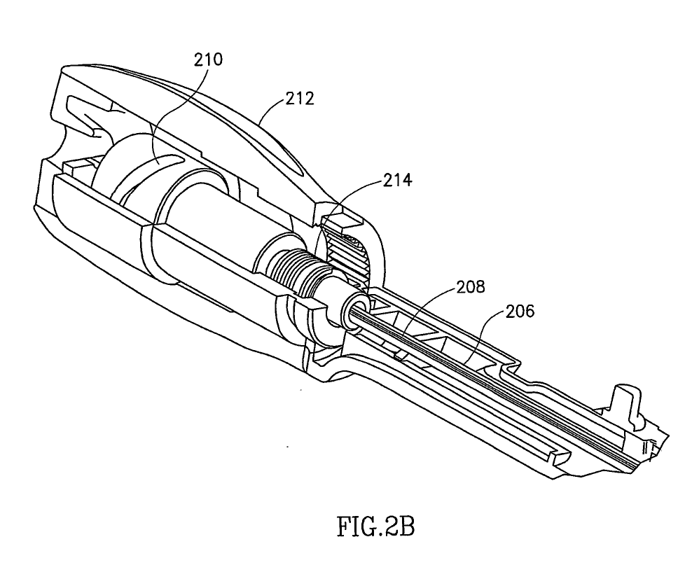

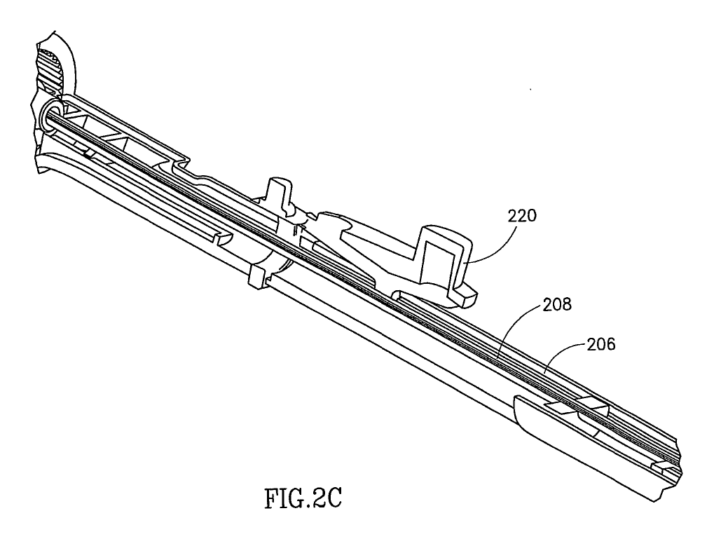

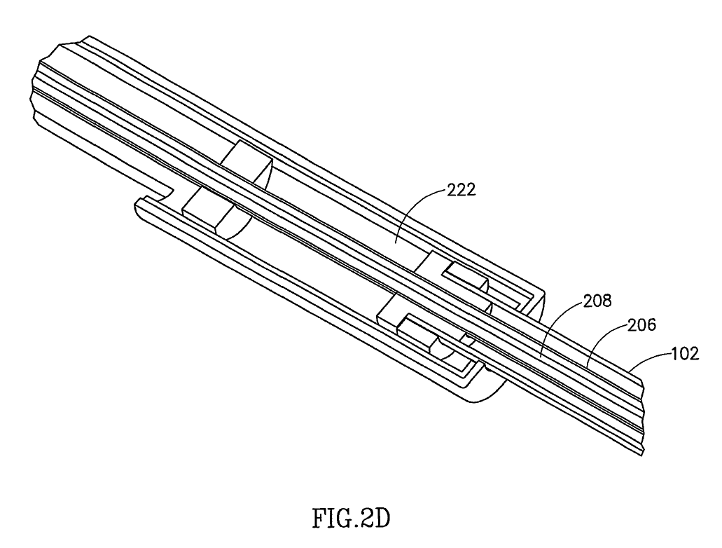

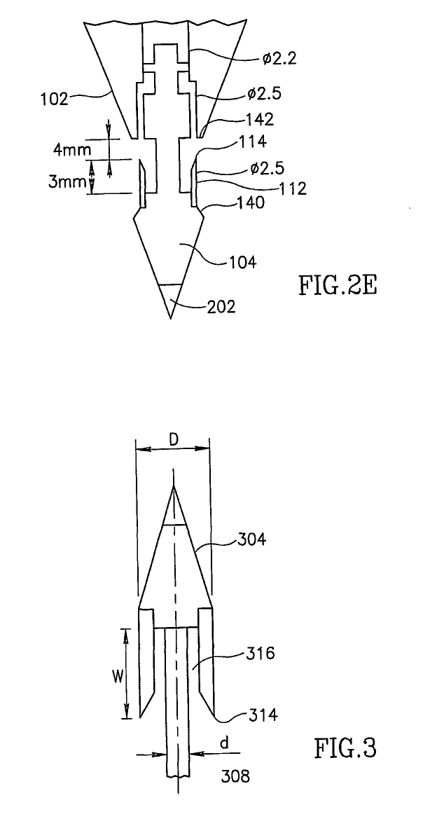

FIG. 1A illustrates a hole former 100 in accordance with an exemplary embodiment of the invention, comprising a base tube 102 and a penetration head 104 for insertion through a wall of a blood vessel 106. As shown, vessel 106 comprises an intima layer 108 and an adventitsia layer 110. As shown for example in FIG. 2, the tip of penetration head 104 may comprises a retractable penetration tip.

In an exemplary embodiment of the invention, penetration head 104 comprises a cutting lip 114 that cuts into vessel 106 when retracted towards the vessel. Optionally, cutting lip 114 is formed as the rim of a cup 116 having a wall 112. Cup 116 desirably serves to contain a tissue plug that is cut out of vessel 106 by cutting lip 114.

In the embodiment of FIG. 1A, base tube 102 defines an anvil surface 118 that contacts cutting lip 114 when penetration head 104 is retracted sufficiently. In an exemplary embodiment of the invention, as it is retracted, lip 114 performs a knife cutting action until it nears anvil 118, where it performs an anvil cutting action, which may be suitable for cutting through adventitsia 110.

FIG. 1B shows an alternative hole former 130, in which the knife cutting action and the anvil action are performed by different surfaces. Wall 112 has an outer diameter smaller than an inner diameter of base tube 102, so that cup 116 can be retracted into a bore 138 of tube 102. If the clearance between lip 114 and bore 138 is small enough, a shearing cutting action can be performed between penetration head 104 and base tube 102. Optionally, lip 114 is sharp enough for performing a knife cutting action.

In an exemplary embodiment of the invention, anvil cutting is provided between a cutting lip 142 of base tube 102 and an anvil portion 140, optionally inclined, of penetration head 134.

Optionally, one or both of penetration head 104 and base tube 102 rotate, in same or in opposite directions. Alternatively to complete rotations, oscillatory rotation is provided.

When retracting penetration head towards base tube 102, one or both of head 104 and tube 102 may be moved. Optionally, for example as described below, the motion is intermittent, allowing an impulse anvil cutting action to be achieved.

Coupling between advancing and rotation is optional. In one example, coupling is achieved by a threading that links advancing to rotation. Alternatively to rotation during retraction, rotation is performed after retraction (e.g., when the edges begin to pinch the vessel wall). Optionally, rotation and retraction are controlled separately, for example using one control for rotation and one for retraction.

FIGS. 2A-2E are cut-through views of an exemplary hole former 200, in accordance with an exemplary embodiment of the invention and similar to the embodiment of FIG. 1B.

FIG. 2A shows an optional retracting penetration tip 202 that is retracted by retracting a shaft 208 to which it is attached after penetration, so that the sharp tip does not damage the far wall of the blood vessel. Optionally, the retraction of the tip unlocks a retraction mechanism that manually or automatically (e.g., using a spring or a motor) retracts the penetration head towards the base section. Also shown is a shaft 206 used for retracting penetration head 104. Former 200 is shown mounted in a delivery system 210, optionally a split delivery system.

FIG. 2B shows a handle section of former 200, which comprises, for example, a rotating handle 212. A slot 210 is used to guide the retraction of penetration tip 202 once the tip penetrates a blood vessel. A threading 214 is used, for example, to control the retraction and rotation of penetration head 104 during use of hole former 200.

FIG. 2C shows a central section of former 200, including an optional clip 220 for locking former 200 into delivery system 210.

FIG. 2D shows a section of former 200 in which base tube 102 is coupled to the rest of former 200. As will be shown below, an optional volume 222 is used to contain a resilient element (e.g., silicon or a spring) that couples base tube 102 to former 200.

FIG. 2E shows exemplary measurements for system 200 for use in a human aorta.

It should be noted that, in an exemplary embodiment of the invention, once the plug is removed from the vessel wall, base tube 102 is advanced into the formed hole, for example, to prevent blood leakage.

FIG. 3 illustrates various dimensions of a penetration head 304 that maybe relevant in accordance with an exemplary embodiment of the invention. A diameter d is the outer diameter of a shaft 308, used to retract head 304. A diameter D is the outer diameter defined by a cutting lip 314. A depth W is a depth of a tissue receptacle area 316 that contains the plug. The inventors have determined that the size of tissue plug removed from the target vessel is dependent on the geometry of the tissue receptacle. Thus, if W is too small, the tissue plug will be restricted in size. Similarly, if D is near d, there is less room for the tissue plug. Optionally, the use of a cutting lip 314 rather than a blunt end ensures that less tissue will slip past, since lip 314 cuts into the tissue and holds it in place. Optionally, the receptacle geometry is designed to affect a certain plug geometry. For example, if the receptacle fills up before cutting is completed, the plug diameter will decrease. The direction of decrease along the thickness of the plug may depend on the direction of cutting and/or receptacle orientation. For example, if the tissue receptacle and/or cutting lips are formed on tube 102, the decrease will be towards the blood vessel. In addition, knife cuts may be used to ensure that earlier cut tissue will have a known diameter, while a shearing cut can be used to ensure that later cut tissue will have a geometry based on available receptacle volume. An hourglass profile may be achieved by cutting from both sides of the vessel towards the middle, while using a limited volume tissue receptacle defined between the two cutting sides.

Various rotation/axial ratios may be used, for example, 1/1—one rotation per mm advance. In one example, at least 10 or at least 30 rotations are provided during a hole forming. In another example, only one, or fewer rotations are provided.

If W is large enough, the tissue plug removed from the body will lodge in receptacle 316 and additional use of the hole forming system will be difficult. In particular, a smooth cutting action may indicate a large value for W, so that the tissue plug is substantially inaccessible form outside. In an exemplary embodiment of the invention, mechanisms to assist in removing the plug are provided.

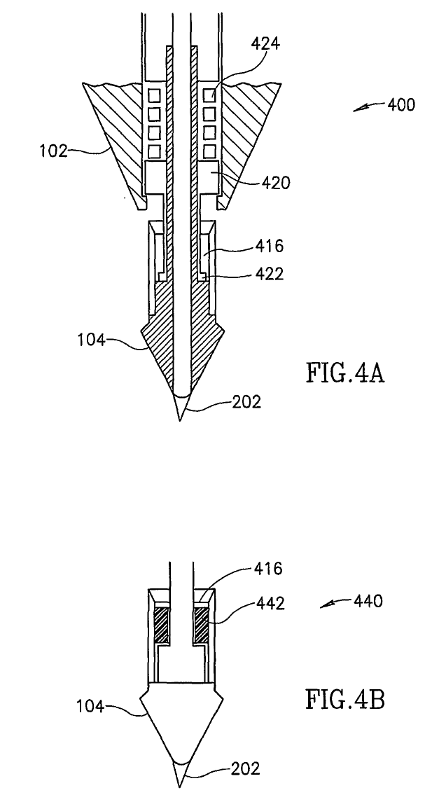

FIG. 4A shows a penetration head 400 in which a tissue extractor 420 is provided for pulling a tissue plug out of a tissue receptacle 416. In an exemplary embodiment of the invention, extractor 420 includes one or more radial extensions (or a lip) 422 that lie inside receptacle 416. When penetration head 104 is advanced, the tissue plug catches on extensions 422 and is extracted from receptacle 416. An optional resilient element 424, for example a spring a soft rubber is provided to allow tissue retractor 420 to be pushed towards base 102. In an alternative embodiment, retractor 420 is free-moving.

FIG. 4B shows an alternative mechanism 440, in which a resilient element 442, such as a spring or a silicon plug is provided in tissue receptacle 416. The resilient element is compressed by the plug during the hole forming operation and rebounds when the operation is complete, to urge out the plug.

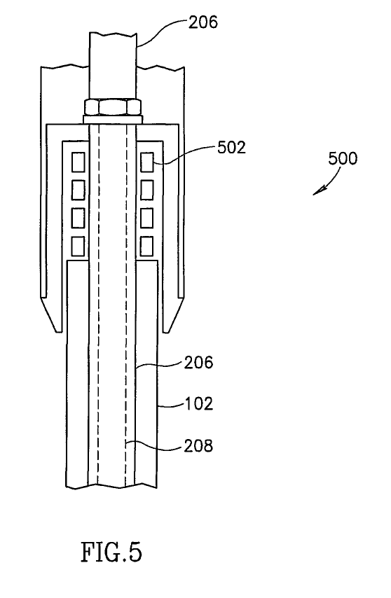

In some embodiments of the invention, for example as shown in FIG. 1A, a cutting lip contacts a non-moving element, and may be damaged thereby. FIG. 5 illustrates a base retraction mechanism 500, in accordance with an exemplary embodiment of the invention, which allows base 102 to resiliently retract. Thus, for example, when contacted by cutting lips, base 102 is pushed back by the lips instead of the lips being ground down. One potential advantage of such resilient contact is that it allows a looser manufacturing tolerance when designing a thread for coupling axial and rotational motion of penetration head 104.

In an exemplary embodiment of the invention, mechanism 500 comprises a resilient element 502 (or base 102 may be made resilient) such as a lump of soft silicon rubber or a spring, that allows some axial motion of base 102.

An additional potential advantage of such resilience is that it allows penetration head 104 to continue rotating after it contacts base 102. An additional potential advantage is that if penetration head jumps a thread after it contacts base 102, this causes an impulse motion of head 104 relative to base 102, which may assist in cutting the adventitsia.

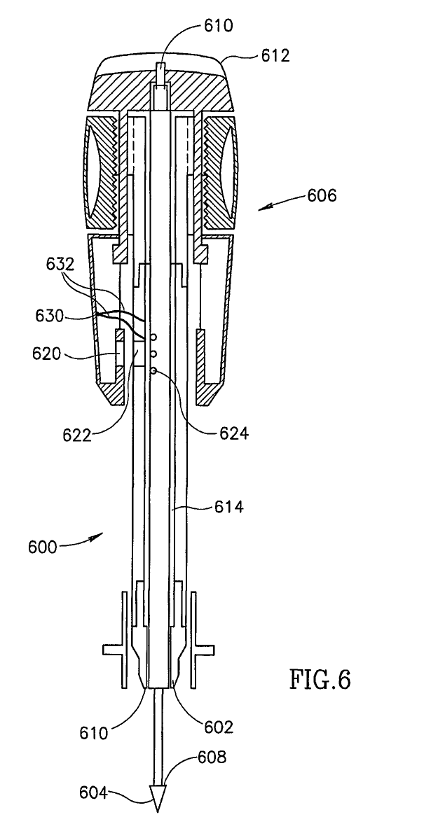

FIG. 6 illustrates an alternative hole former 600, in accordance with an exemplary embodiment of the invention. In this embodiment, former 600 comprises a penetration head 604 with an optional retracting penetration tip (not shown). Slicing action is optionally provided between the upper edge of penetration head 604 and the inner diameter of a base 602. Alternatively or additionally, knife cutting action is provided by an inner lip 608 of penetration head and/or a forward lip 610 of base 602. One or both of head 604 and base 602 rotate. Optionally, head 604 is retracted using a threaded drive actuated in handle 606. Alternatively, head 604 (and similarly heads on other embodiments described herein) may be retracted using a spring loaded mechanism.

Also useful, as illustrated for example, in FIG. 6, are various marking systems for indicating the progress of hole forming. One exemplary system comprises an aperture (or transparent portion) 620 defined in handle 606 and a second aperture 622 formed in base 602. One or more visual markings 624 on a shaft 614 that is coupled to penetration head 604 may be visible through the apertures/transparent sections to indicate a relative location of penetration head 604 and base 602.

Another exemplary indication system comprises a transparent dome 612 through which is visible the extension of a bar 610 (which extends as penetration head 604 is retracted), is visible.

Another exemplary system is an electrical system in which references 624 indicate contacts (rather than markings) on shaft 614 short together leads 632 to allow a battery (not shown) to power light 630, a LED for example. This allows the indication to be better located than using mechanical means. Alternatively or additionally, a mechanical (or electrical) sound, such as a click is sounded when the retraction of head 604 is completed. Possibly, different sounds are generated during retraction and after head 604 contacts base 602. Alternatively or additionally, a resistor and slide arrangement is used to indicate progress on a meter other suitable scale display.

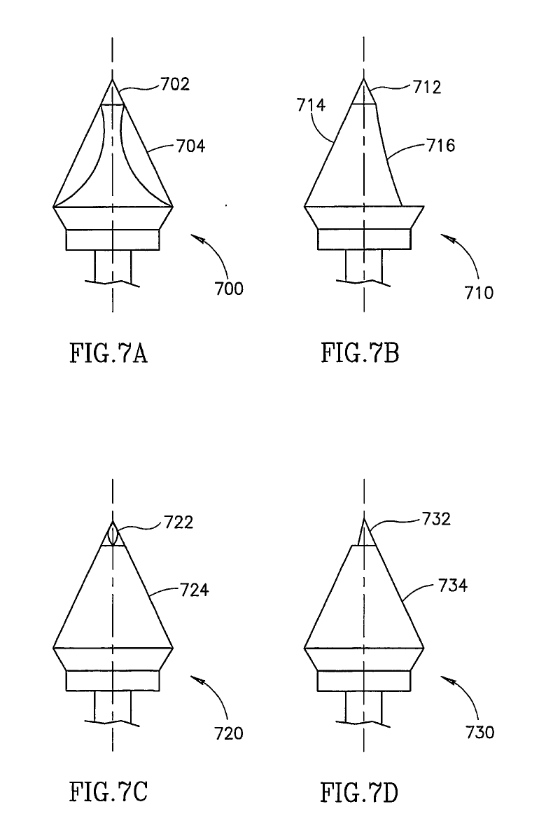

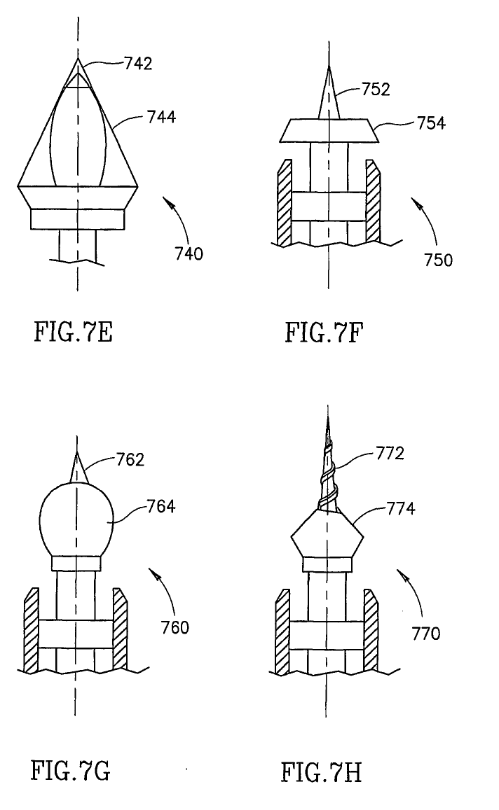

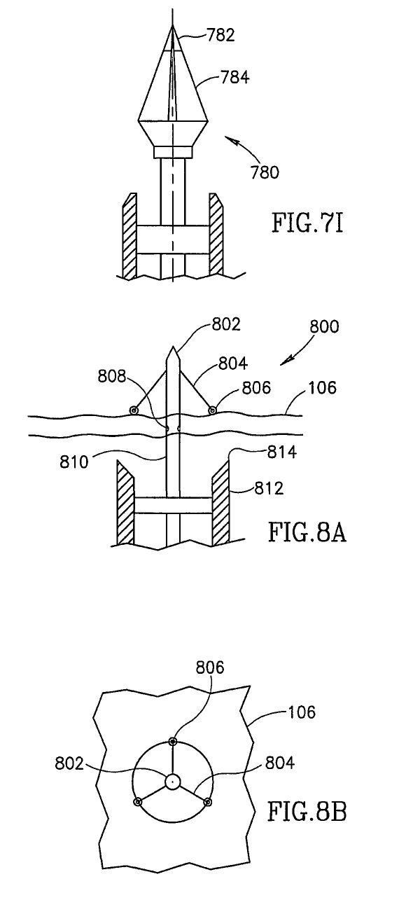

FIGS. 7A-7I illustrate various penetration tip and penetration head designs, in accordance with exemplary embodiments of the invention. The penetration tips are optionally retractable in each of the diagrams shown.

FIG. 7A shows a penetration head 700 including a head body 704 that is deeply scalloped on one, two, three or more sides and a penetration tip 702, that is conical.

FIG. 7B shows a penetration head 710 including a head body 714 that is asymmetric and sharpened along one edge 716 thereof and having a matching knife shaped penetration tip 712.

FIG. 7C shows a penetration head 720 including a conical head body 724 and a penetration tip 722, that is scalloped.

FIG. 7D shows a penetration head 730 including a conical head body 734 and a penetration tip 732, that is a one sided knife.

FIG. 7E shows a penetration head 740 in which scalloping on a head body 744 matches scalloping on a penetration tip 742.

FIG. 7F shows a penetration head 750 in which a head body 754 is a truncated cone having a longer and sharper penetration tip 752, for example, having a length that is 2 or three times its diameter.

FIG. 7G shows a penetration head 760 in which a head body 764 is bulbous and blunt, with a regular penetration tip 762.

FIG. 7H shows a penetration head 770 in which a head body 774 is associated with a threaded penetration tip 772 that is optionally rotated as it is advanced.

FIG. 7I shows a penetration head 780 in which a head body 784 and its associated penetration tip 782 are formed in the shape of a knife having the cross-section of a cross.

Other variations are contemplated as well, for example, one or both of the cutting lips on the penetration head and base 102 may be oblique relative to the axis or relative to the radius of the system (e.g., have a non-constant radius). Such oblique elements may be provided, for example, for embodiments with inner lip cutting or with outer lip cutting. The different parts may have different degrees of obliqueness.

Optionally, for any embodiment of the invention with an asymmetric penetration head or tip, such as those shown in FIGS. 7A-7E, and 7I there are markings further back on the hole former so that the surgeon can easily see how the penetration head or tip is oriented. Often there is an optimal angle of orientation for an asymmetric penetration head or tip with respect to the axis of the blood vessel, to minimize tearing for example.

FIGS. 8A and 8B illustrate an expanding penetration head 800, in accordance with an exemplary embodiment of the invention. Head 800 comprises a penetration tip 802 mounted on a shaft 810. A plurality of arms 804 extend radially at an angle from shaft 810. Optionally, the arms are contained in slots 808 defined in shaft 810. In an exemplary embodiment of the invention, the arms spring out when shaft 810 exits a confining outer base tube 812 and after it passes through the confinement of a wall of vessel 106. In an exemplary embodiment of the invention, arms 804 end in rounded tips 806. FIG. 8B shows a top view of FIG. 8A. Optionally, arms 804 are slivers formed out of the body of shaft 810.

In use, shaft 802 is retracted relative to base portion 812. Cutting action may be achieved by a cutting edge 814 of tube 812. Alternatively or additionally, tips 806 serve as a partial anvil for urging tissue against cutting edge 814. Optionally, shaft 802 and/or base 812 are rotated.

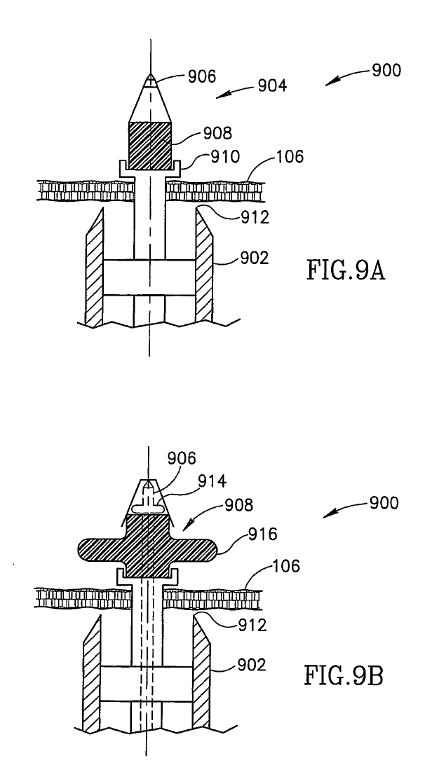

FIGS. 9A and 9B illustrate an alternative expanding penetration head 904, in accordance with an exemplary embodiment of the invention. A hole former system 900 comprises a base tube 902 having a cutting edge 912 and an expanding head that has a small diameter when inserted through a vessel 106 (FIG. 9A) and a larger diameter during hole forming (FIG. 9B). In an exemplary embodiment of the invention, head 904 comprises a resilient and/or expandable element 908, for example comprising silicon or other fluid or semi-fluid material, that is deformed and caused to expand out so that extensions 916 (or a disc) are formed. In an exemplary embodiment of the invention, A penetration tip 906 of head 904 (and optionally an associated base 914) or the whole of head 904 are retracted relative to a base portion 910 of head 904, this causes the silicon element 908 to be axially compressed and radially extend. Alternatively, element 908 may be expanded or it may be deformed by the advancement of a rod into the element from the direction of tube 902.

In an exemplary embodiment of the invention, extensions 916 serve to urge the wall of vessel 106 towards base 902. Alternatively or additionally, extensions 916 serve as an anvil for cutting edge 912. Optionally, silicon element 908 has one or more hard patches on its surface. In an exemplary embodiment of the invention, such hard patches can be used for the anvil cutting action, however, they are not required. Alternatively or additionally, extensions 916 fit inside base tube 902 and provide for shearing cutting action. Alternatively or additionally, the expansion of element 908 causes one or more sharp spikes or cutting edges (not shown) to extend in the direction of base 102. Optionally, extensions 916 are inclined at the point of contact with cutting edge 912, providing for an angular anvil cutting action. Optionally, the resilience of element 908 is such that when cutting edge 912 meets/nears extensions 916, the extensions give, allowing a sliding of edge 912 relative to extensions 916.

It should be noted that even a soft anvil or scissors part can provide some benefits over a free cutting action. In addition, the resiliency of the silicon can be manipulated (during manufacture) to provide a maximum hardness that still allows the silicon to be deformed.

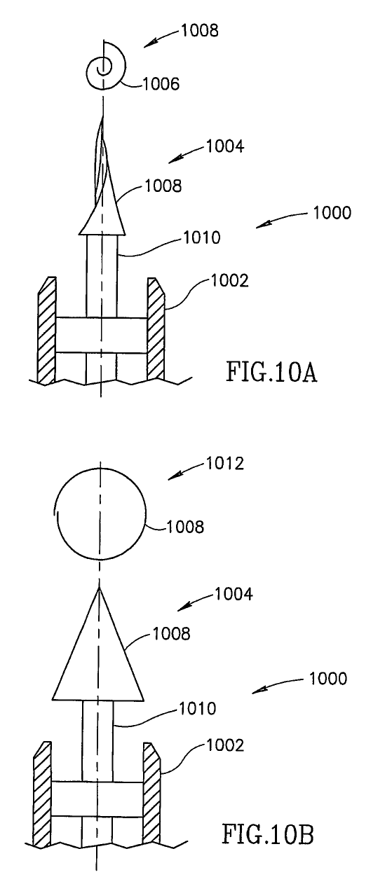

FIGS. 10A and 10B illustrate a hole former 1000 that includes an expanding penetration head 1004, in accordance with an exemplary embodiment of the invention.

In an exemplary embodiment of the invention, head 1004 comprises a thin sheet 1008 that is tightly wound around its axis, as shown in a cross-section 1006. FIG. 10B shows former 1000 after deployment, when head 1004 is released to achieve a conical shape. A cross-section is shown as reference 1012. A shaft 1010 is optionally welded to the side or to the tip of head 1004. Alternatively, sheet 1008 is manufactured out of shaft 1010.

Once head 1004 expands, head 1004 may be retracted towards a base tube 1002 to provide for cutting action, for example, knife, shearing and/or anvil cutting action, as described herein, depending, inter alia, on the relative geometry of head 1004 and base 1002.

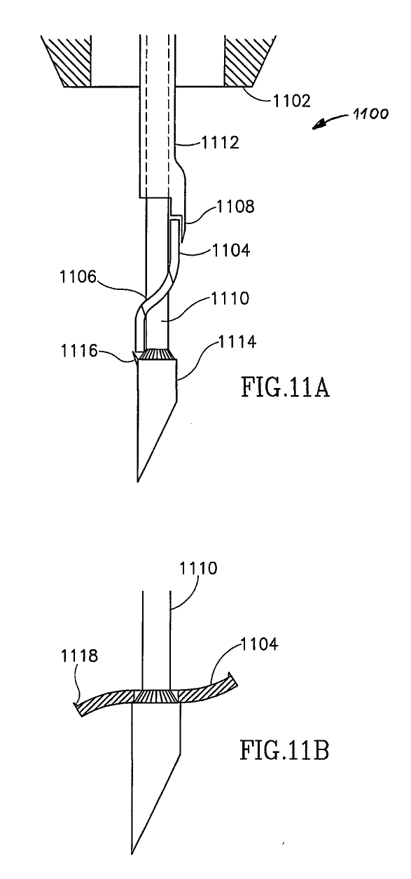

FIGS. 11A and 11B illustrate a hole former 1100 including a geometry changing anvil 1104, in accordance with an exemplary embodiment of the invention. Hole former 1100 includes a penetration tip 1114 mounted on a shaft 1110 and a base tube 1102. A cut-assisting disk 1104, optionally having an aperture 1106 is mounted on shaft 1110. In an exemplary embodiment of the invention, an over tube 1112 (or other similar restraining element) maintains disk 1104 in a distorted configuration, for example, the disk being held between an extension 1108 of tube 1112 and shaft 1110. Optionally, a second extension 1116, holds another portion of disk 1104 against penetration tip 1114.

In FIG. 11B, penetration tip 1114 and disk 1104 are inserted through a blood vessel wall and tube 1112 is retracted, thus freeing disk 1104 to achieve an orientation perpendicular to shaft 1110. Disk 1104 can now be used as an anvil or as a shearing base, depending, inter alia, on the relative geometries of disk 1104 and base 1102. Optionally, disk 1104 includes one or more spikes or a cutting edge 1118, so that it can be used for cutting. Optionally, aperture 1106 of disk 1110 has a geometry that mates the cross-section of shaft 1110, preventing rotation.

In an exemplary embodiment of the invention, disk 1104 is aligned with a direction of a cut formed by penetration tip 1114. Alternatively or additionally, disk 1104 has a sharp edge that assist in forming a cut.

Optionally, disk 1104 is made oblique by the distortion, so that its trans-axial dimension is small. Alternatively or additionally, disk 1104 is always oblique. Alternatively or additionally, disk 1104 is maintained in a distorted configuration by tension, between one part that is held by the penetration tip 1114 and another part that is held back by over tube 1112.

Alternatively or additionally, disk 1104 is plastically distorted, for example, by the advance of over tube 1112 flattening disk 1104. Alternatively or additionally, disk 1104 is bi-stable between the configurations of FIGS. 11A and 11B.

In this and in other embodiments, various shape changing mechanisms may be used, for example, the above mentioned shape changing mechanism and elastic, super-elastic and shape-memory based distortion.

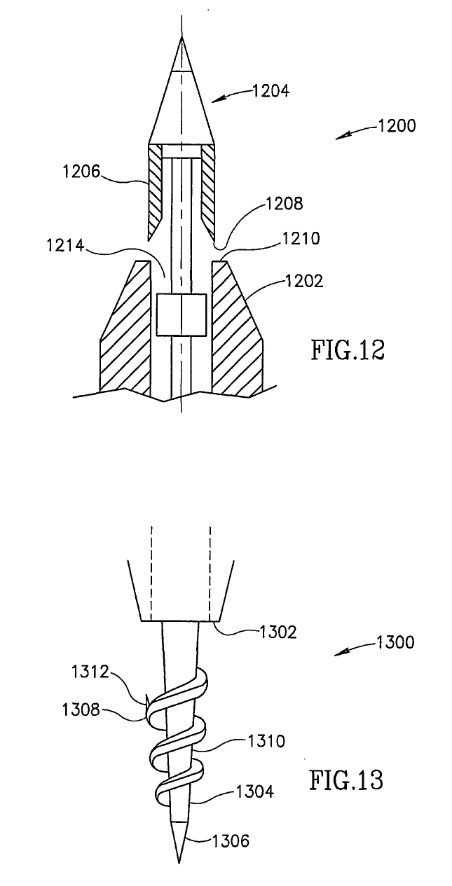

FIG. 12 illustrates a resilient anvil hole former 1200, in accordance with an exemplary embodiment of the invention. Former 1200 comprises a penetration head 1204, for example as described above, which includes a wall 1206 having a cutting edge 1208. A base 1202 is also provided, however, unlike some of the embodiments described above, base 1202 has a front end 1210 that is resilient. In one embodiment, cutting edge 1208 can penetrate into front end 1210. In another embodiment, cutting edge 1208 compresses end 1210 and then optionally slides into an hollow axis 1214 defined by the distorted base 1202. Optionally, the degree of resilience is selected to assist in cutting adventitsia tissue.

FIG. 13 illustrates a hole former 1300 including a thread-type penetration head 1304, in accordance with an exemplary embodiment of the invention. Head 1304 comprises a shaft 1310 on which a threading 1308 is provided. Optionally, a retractable penetration tip 1306 is provided. In use, shaft 1310 is inserted through a blood vessel wall and then rotated to advance the shaft using the threading. Once some or all the threading is through the wall, penetration head 1304 is retracted towards a base 1302, to cut the wall tissue. In one example a cutting edge 1312 is provided on thread 1308. Alternatively or additionally, a shearing cutting action is performed between a thread turn and base 1302.

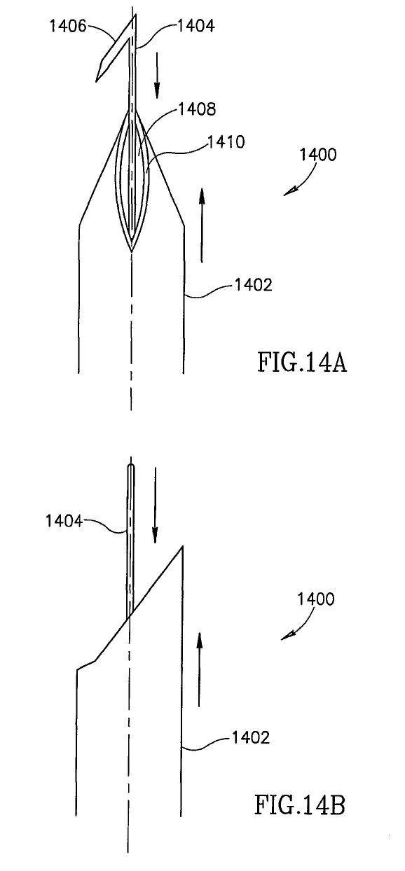

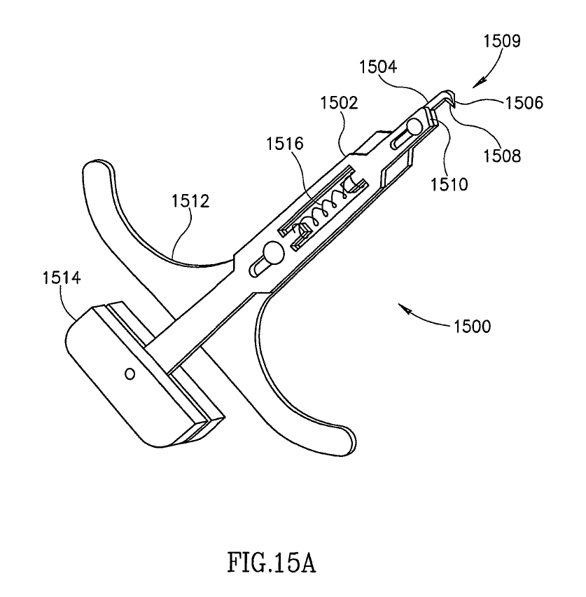

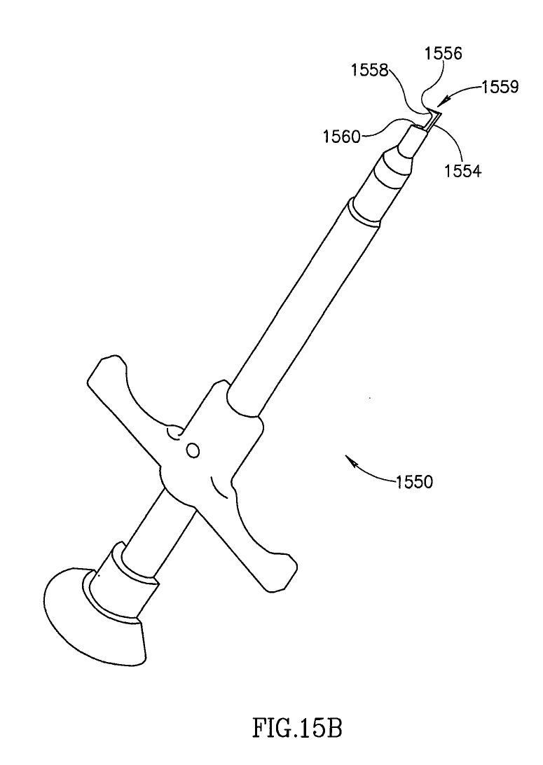

FIGS. 14A and 14B are perpendicular side views of a needle-type hole former 1400, in accordance with an exemplary embodiment of the invention. A hollow pointed needle 1402 is formed with an oblique aperture 1408 optionally having a sharpened cutting lip 1410. In use, a penetration tip 1404 is extended through a wall of a blood vessel and then retracted towards the needle. In an exemplary embodiment of the invention, tip 1404 includes an extension 1406, for example an elastically extending extension that extends once the penetration tip passes out of the needle and through the tissue. Optionally, extension 1406 serves as a knife. Alternatively or additionally, the tip of extension 1406 is inserted into the target blood vessel first and then turned, for example as in the embodiment of FIG. 15.

FIGS. 15A and 15B illustrate two variants of an incision maker, in accordance with an exemplary embodiment of the invention. FIG. 15A shows an incision maker 1500. Two moving parts are provided, a base face 1510 coupled to a first handle 1514 and an “L” shaped spike 1504 coupled to a second handle 1512. Other handle designs may be used. The two parts are optionally coupled using a spring 1516. In use, a tip 1506 of an arm 1509 of spike 1504 is inserted into a blood vessel, for example a coronary artery. Incision maker 1500 is then turned so that arm 1509 is inside the vessel and parallel to the vessel axis (assuming that is the desired cut direction, as an oblique cut or a trans-axial cut may be desired). Arm 1509 is then retracted towards face 1510 and the vessel wall is cut using a shearing cut. Optionally an inner face 1508 of arm 1509 is sharp and functions as a knife.

FIG. 15B shows an alternative embodiment of an incision maker in accordance with the invention, in which two base faces 1560 are provided, one on either side of a spike 1554 (only one face is visible). A spike tip 1556 of an arm 1559 and an optionally cutting edge 1558 of arm 1159 may function as before.

Optionally, face 1560 and arm 1559 while optionally in substantially parallel are not parallel to each other, for example, spreading out (as shown) or pointing in.

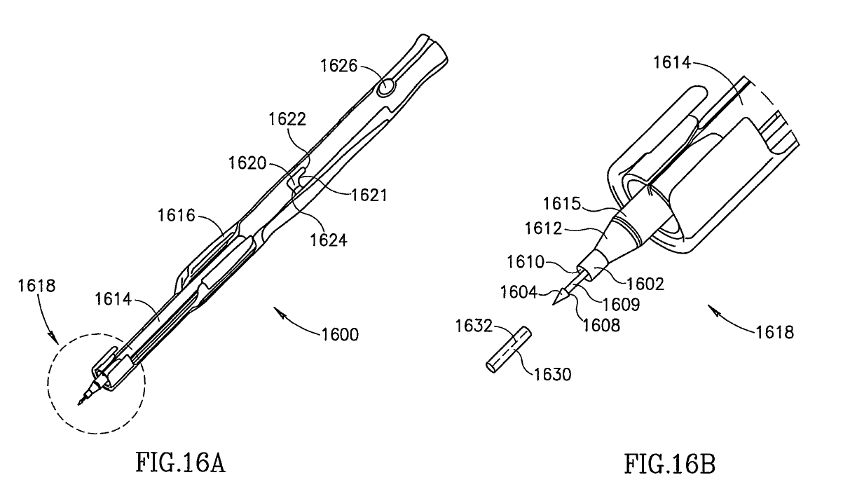

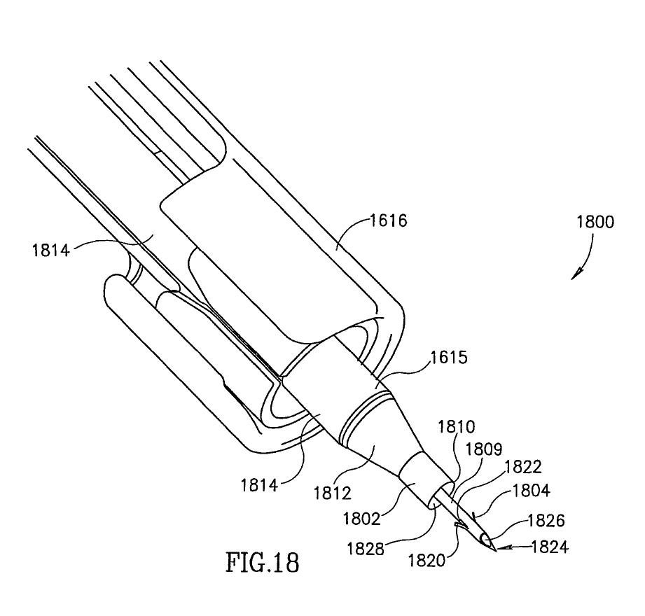

FIGS. 16A and 16B illustrate a hole former system 1600 in accordance with an alternative embodiment of the invention. FIG. 16A shows former 1600 in a scaffold delivery system 1616 and FIG. 16B shows an enlargement of a tip 1618 of former 1600. Referring first to FIG. 16B, former tip 1618 comprises a sharp penetration head 1604 adapted to be inserted into a blood vessel, so that a shaft portion 1609 of penetration head 1604 transfixes the blood vessel wall. Optionally, head 1604 includes a roughened surface, barbs, threads, a tissue receptacle (e.g., 116 of FIG. 1) or a widening 1608 (such as the cone shape shown), to prevent tissue from falling off head shaft 1609, as described in more detail below. In an exemplary embodiment of the invention, angled extensions are formed out of a straight shaft by cutting into the shaft at an angle at several locations (e.g., 2 or 3) and pulling or curling the cut sections out in a radial direction, for example as shown in FIG. 18 below.

Cutting of the target vessel is achieved by a cutting surface 1610 formed on a base section 1602, for example a tube. As noted above, the cutting surface may be smooth, jagged, serrated and/or wave-like, possibly different finishes on different parts of the surface. Optionally, cutting surface 1610 defines an oblique surface relative to shaft 1609 or is not all in one plane. Base 1602 is optionally connected to a shaft 1614 of former 1600, using an inclined section 1612, which may be used for assisting in advancing a sleeve 1615 of scaffold 1616 into a formed aperture in a blood vessel.

Optionally, penetration head 1604 is locked to base section 1602, during cutting, to prevent its axial motion and optionally also its rotational motion.

In an exemplary embodiment of the invention, after a hole is cut using surface 1610, penetration head 1604 is retracted pulling a plug of tissue that is cut out into a lumen in base 1602. Optionally, the retraction is manual. Alternatively, the retraction is spring loaded. Alternatively, other power sources may be used for retraction, for example, pneumatic power, such as available at gas pressure outlets in many hospital rooms. In another example, an electrical motor or solenoid is used to retract penetration head 1604. The retraction may be wholly axial or it may include a rotational component. In some embodiments of the invention, penetration head 1604 has rotational freedom relative to base 1602, while in other embodiments it is rotationally fixed. Base 1602 may or may not rotate relative to scaffold 1616.

In an exemplary embodiment of the invention, a peg 1620 is provided in a channel 1621 which has two resting spots, the position of peg 1620 as shown in FIG. 16A (1624), where head 1604 is extended and a position 1622 at which head 1604 is retracted. Optionally, a safety release switch 1626 is provided to lock head 1604 and prevent axial motion of head 1604 relative to base 1602 and/or to lock the hole former 1600 in delivery scaffold 1616.

The use of a general scaffold 1616 with which different tools can be delivered is not crucial for carrying out the invention. However, some types of such scaffolds include an inner leaflet valve through which the tools are advanced. In some cases, surface 1610 and/or head 1604 may damage the valve when the hold former is advanced through the scaffold. In an exemplary embodiment of the invention, a protective covering 1630 is provided. In an exemplary embodiment of the invention, covering 1630 comprises a tube, for example, a silicone tube or a shrink-fitted tube that isolates the valve from the sharp edges of former 1600 (or other tool), for example, surface 1610 and the tip of head 1604. After insertion, covering 1630 is torn off or pulled off (e.g., if it has one sealed end. Optionally, covering 1630 includes a perforation 1632, a rip cord and/or a pull tab, to assist in removal after it is inserted in scaffold 1616.

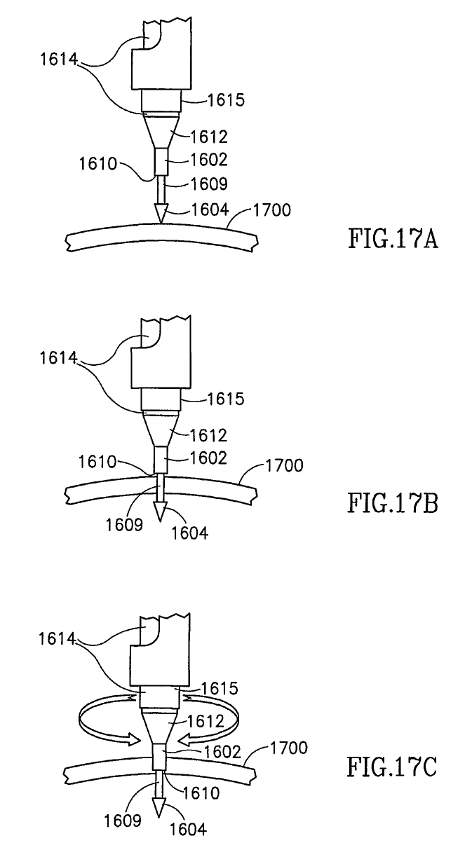

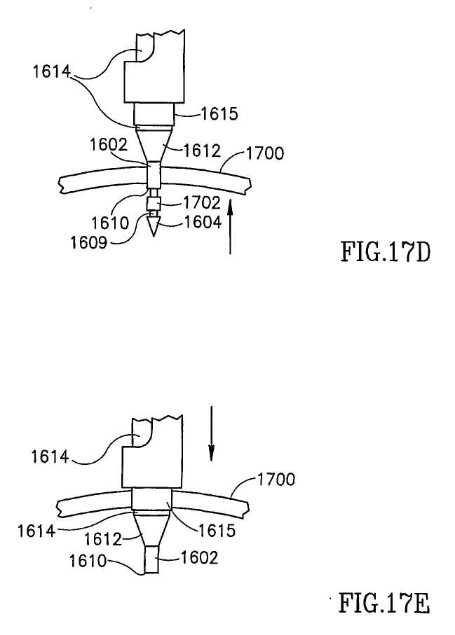

FIGS. 17A-17E illustrate the use of hole former 1600, in accordance with an exemplary embodiment of the invention.

In FIG. 17A, penetration head 1604 is advanced towards a blood vessel, for example an aorta 1700.

In FIG. 17B, penetration head 1604 is advanced to penetrate vessel 1700, so that shaft 1609 transfixes vessel 1700 and penetration head 1604 does not engage vessel 1700 in any way. In some embodiments, however, penetration head 1604 includes barbs for engaging vessel 1700 or remains inside the wall of the vessel. Such engagement may cause the vascular tissue to be stretched before being cut, possibly providing apertures that are smaller or larger than the diameter of base 1602 and/or have a conical profile. The size and shape may depend on whether penetration head 1604 is retracted prior to cutting starting and/or being completed. Optionally, penetration head 1604 includes a retracting sharp tip (e.g., FIG. 4A).

In FIG. 17C, cutting is performed, for example, by rotating and/or advancing base 1602 relative to vessel 1700, so that cutting surface 1610 cuts into vessel 1700. Depending on the implementation of former 1600, the entire delivery system may be moved/rotated or only base 1610 and/or other sub-components of system 1600 are rotated and/or moved.

In FIG. 17D, cutting is complete, so base 1610 is engaged by vessel 1700, while a plug 1702 of tissue remains on shaft 1609. Possibly, some or all of plug 1702 is contained inside base 1602. Optionally, a tissue receptacle (not shown) is provided on penetration head 1604.

Penetration head 1604 is retracted, pulling along with it plug 1702, into a lumen formed in base 1602. Penetration head 1604 optionally has significant clearance relative to the inner diameter of the lumen. Alternatively, a small clearance is provided, so that base 1602 and penetration head 1604 can exhibit a shearing action between them (e.g., to cut any loose strands). Optionally, penetration head 1604 is retracted prior to the cutting being completed, but in a the embodiment pictured, it is not so retracted. Alternatively, penetration head 1604 is retracted while base 1602 is advanced, for example to ensure that it does not damage the far side of the blood vessel. Optionally, however, penetration head 1604 is retracted in a manner that ensures that penetration head 1604 does not apply tension or undue tension on vessel 1700, and affect the aperture cutting shape. In one example the penetration head is retracted such that the distance between penetration head 1604 and base 1602 is greater than the thickness of plug 1702, or at least an uncut thickness thereof.

It should be noted that if vessel 1700 is filled with blood under pressure, there is little danger of penetration head 1604 damaging the far side of vessel 1700, especially if the length of shaft 1609 and penetration head 1604 is considerably less than the diameter of vessel 1700. Alternatively, a retracting penetration tip is provided. Desirably surface 1610 is advanced under light pressure, possibly under its own weight, to prevent distortion of vessel 1700. Alternatively, vessel 1700 may be kept in shape by pressure (e.g., with fingers or a tool) on its sides that are perpendicular to the penetration.

In FIG. 17E, the entire hole former is advanced, so that sleeve 1615 enters the wall of vessel 1700 and the hole forming mechanism can be removed. An anastomosis delivery system may now be provided through scaffold 1616 and its valve.

In an exemplary embodiment of the invention, shaft 1609 has a length greater than the thickness of the wall of vessel 1700, for example, being 150%, 200% or 300% its thickness. In an aorta, this translates, for example, into a length of 4-6 mm. Alternatively, the shaft may be shorter than a vessel diameter. Optionally, different length shafts are provided for different patients and/or vessel sizes. Alternatively, a screw or other mechanism is used to adjust the length of shaft 1606, for example, by controlling the resting location of peg 1620. The diameter of penetration head 1604 may be selected to be the diameter that prevent sliding off of plug 1702, while allowing clearance relative to base 1602. The relation between the diameter of shaft 1609 and cutting surface 1610 is optionally as defined in FIG. 3.

FIG. 18 illustrates a tip of a hole former 1800 in accordance with an alternative embodiment of the invention. Former 1800 comprises a shaft 1814 coupled by a cone 1812 to abase section 1802 having a cutting lip 1810 and an inner lumen having a surface 1828. A penetration head 1804 comprises a needle like shaft 1809 having formed out of its body one or more barbs 1820, cut out of depressions 1822. Other methods of forming and attaching such barbs may be used as well. Optionally, shaft 1809 has a needle like tip 1824 with an optional inner lumen having an inner surface 1826.

In an exemplary embodiment of the invention, barbs 1820 are elastic, so that when inserting head 1804 into vessel 1700, barbs 1820 bend back into depressions 1822 and present a smaller resistance to insertion. After insertion, the spring out again.

Optionally, surface 1826 and/or surface 1828 have inner threads, barbs or other treatment, to better engage tissue plugs. Alternatively, the inner diameter of the lumens vary, for example, non-monotonicly, or monotonicly increasing (away form the blood vessel).

A hollow tip such as provided in FIG. 18 may have other uses as well, for example, for eluting medication (e.g., against clotting, for healing the cut tissue and/or to assist in cutting), for example, continuously or when a suitable control (e.g., attached to a reservoir) is used. Alternatively or additionally, such a lumen is used for providing vacuum to better couple former 1800 and vessel 1700. Alternatively or additionally, vacuum is provided between penetration head 1804 (if any) and base 1802, e.g., through the lumen in base 1802. Alternatively, eluting of medication may be provided in other ways, for example, by penetration head 1804 being spongy or from base 1802, for example, from its lumen or its walls.

In an alternative embodiment of the invention, no penetration head is provided, with tissue plug 1702 optionally prevented from falling off by inner threading of surface 1828 of base 1802. Optionally, however, an axial stabilizer like penetration head 1804 and shaft 1809 are provided. In one example, a wire is provided. Alternatively, a spiral cork-screw like shaft 1809 is provided. This inner stabilizer may or may not have a fixed axial position relative to base 1802. If not fixed, the range of motion may nevertheless be fixed and/or the number of stable positions be limited. In an exemplary embodiment of the invention, the stabilizer is fixed so that it protrudes by a large amount (e.g., 1-5 mm for an aorta), slightly (e.g., 1 mm), is even with or is retracted relative to a plane defined by surface 1810. Optionally, the stabilizer is not strong enough (or does not engage vessel 1700 well enough) to be used to urge vessel 1700 against base 1802.

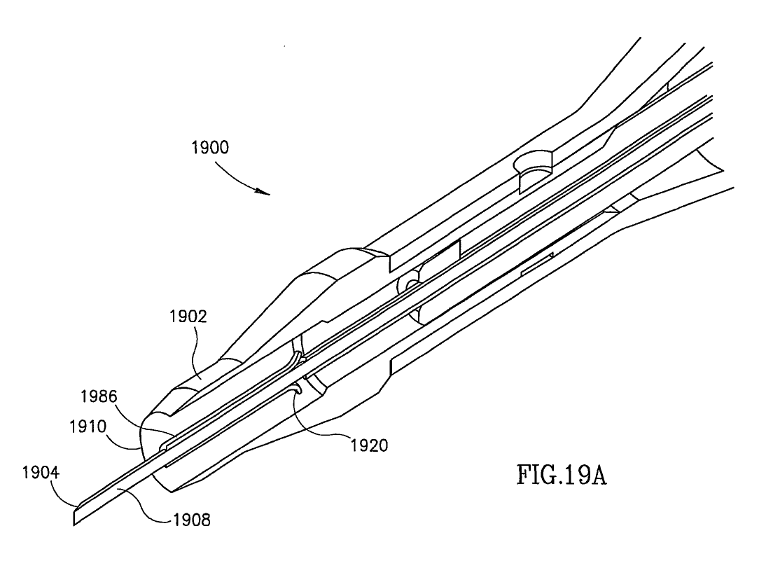

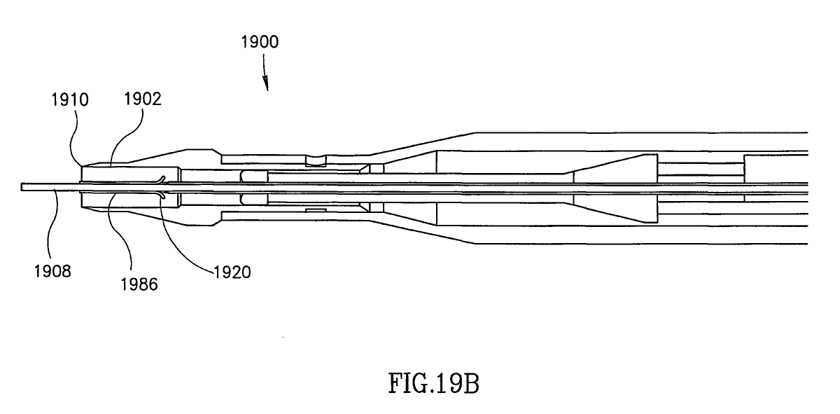

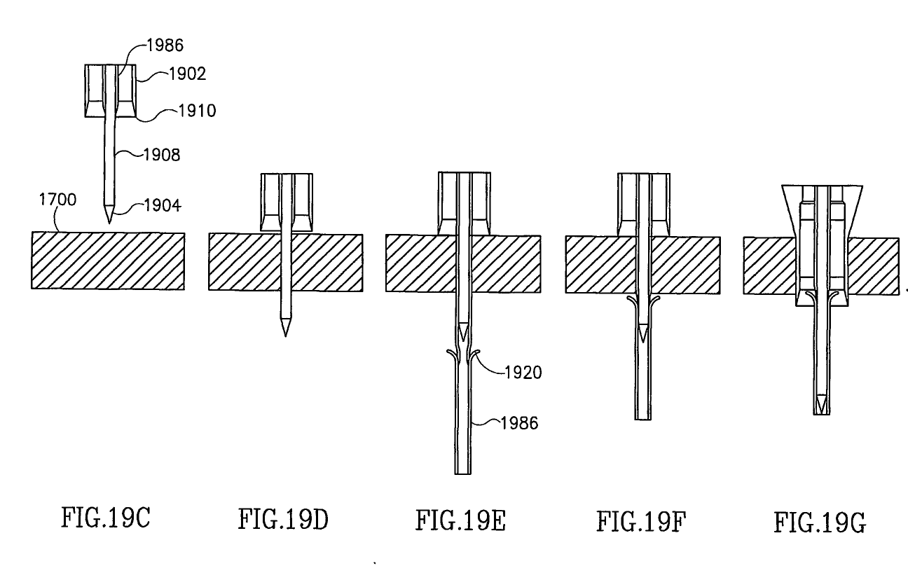

FIGS. 19A and 19B show a hole former 1900 according to another exemplary embodiment of the invention, and FIGS. 19C-19G illustrate how this hole former works, in five steps. In this embodiment of the invention, there are separately manipulated tissue penetrating and tissue holding elements, and the blood vessel wall can be penetrated without holding it, making it possible, for example, for the surgeon to withdraw the penetration element with little damage to the blood vessel, before holding the tissue.

A penetration tip 1904, at the end of an inner penetration shaft 1908, penetrates the blood vessel wall 1700 in FIG. 19D, and a base 1902 is then brought against the outside of the blood vessel. Alternatively, base 1902 is brought against the outside of the blood vessel even before penetration tip 1904 starts to penetrate the wall, or only later, in the stage shown in FIG. 19F, when the surgeon is ready to cut a hole in the wall. An outer penetration shaft 1986, fitting closely around inner shaft 1908, then (in FIG. 19E) passes through the small opening made in the wall by the penetration tip, guided by inner shaft 1908. Until outer shaft 1986 enters the blood vessel, the process is completely reversible with little damage to the blood vessel. For example, if the surgeon decides that a better location should be chosen, then he can withdraw shaft 1908 without pulling the blood vessel, and only minimally damaging the wall. Even if penetration tip 1904 has already reached the inside of the blood vessel, the hole it makes will be much smaller than if the hole former were torn out with the tissue held, and such a small hole is often self-sealing.

After inner shaft 1908 has penetrated the wall completely in a satisfactory way, outer shaft 1986 passes through the wall also. Optionally, outer shaft 1986 rotates as it advances through the wall, and optionally it has a sharp lip at its end, so that it actually cuts the wall as it advances, enlarging the opening made by inner shaft 1908. Alternatively, outer shaft 1986 enlarges the opening by punching. Alternatively outer shaft 1986 is blunt at its end, and it only pushes the wall tissue out of the way, or dilates the tissue, as it passes through the opening made by inner shaft 1908.

Optionally, penetration tip 1904 and penetration shaft 1908 are retracted into outer shaft 1986 once outer shaft 1986 is through the wall, to reduce the risk of the penetration tip inadvertently damaging the other side of the blood vessel. Optionally, outer shaft 1986 has a small balloon or another deployable blunt element at its end, or deployed through its lumen, to keep the end of outer shaft 1986 from inadvertently damaging the opposite wall of the blood vessel. Alternatively or additionally, inner shaft 1908 has such a deployable blunt element near its end, or inside it. Optionally, penetration tip 1904 is retractable into shaft 1908, so that tip 1904 can be retracted any time after the end of shaft 1908 is through the wall.

Alternatively, penetration tip 1904 and/or shaft 1908 has a fixed location and cannot be axially retracted. Outer shaft 1986 is optionally made long enough so that even when retracted as far as expected (e.g., an tissue holding element as described below is brought to or into base 1902) during a cutting procedure, tip 1904 remains covered.

In an exemplary embodiment of the invention, outer shaft 1986 has one or more tissue holding elements 1920 on its side, for example projecting elements such as barbs, and once barbs 1920 have passed through the wall (and shaft 1986 has been drawn back somewhat), the barbs prevent shaft 1986 from pulling back through the wall, as shown in FIG. 19F. Optionally, barbs 1920 are flexible and are pressed against the side of shaft 1986 by the tissue, until they get through the wall. Alternatively, barbs 1920 are rigid. When inserted, the barbs may push the sides of the opening away as they pass through the opening, or dilate the tissue, or cut through the sides of the opening as they pass through it. Optionally, instead of barbs, a disk or another kind of projecting element is used, similar to the disk shown in FIG. 11.

Alternatively, instead of having the barbs attached to outer shaft 1986, the barbs are flexible and are attached to shaft 1908, and they are covered by shaft 1986, which presses them against the sides of shaft 1908, until the barbs on shaft 1908 (with shaft 1986 covering them) are through the wall. Then, shaft 1986 is withdrawn enough to uncover the barbs, which project outward, and prevent shaft 1908 from pulling back through the wall. Alternatively, shaft 1986 is withdrawn when the barbs are still in the middle of the wall, and the sides of the opening hold the barbs against shaft 1908 until the shaft 1908 carries the barbs through the wall. Optionally, instead of uncovering the barbs by withdrawing shaft 1986 axially, shaft 1986 has slots in it, and the barbs are uncovered by rotating shaft 1986 azimuthally until the slots are aligned with the barbs. Alternatively, the barbs are uncovered when shaft 1986 moves axially so that the barbs are aligned with the slots, or shaft 1986 moves both axially and azimuthally to align the barbs with the slots. Optionally, there are three shafts, a penetration shaft 1908 without barbs, a shaft 1986 with flexible barbs on it, and an outer shaft which keeps the flexible barbs pressed against shaft 1986 until the outer shaft is withdrawn.

Alternatively, shaft 1908, with penetration tip 1904 at its end, is hollow, and shaft 1986, with barbs 1920 attached to it, is inside shaft 1908. In this embodiment of the invention, shaft 1908 has slots in it, which barbs 1920 fit through. Optionally, the barbs are flexible, and are pressed against the sides of shaft 1986 by the inside of shaft 1908. When shaft 1908, including the slots, has passed through the vessel wall, shaft 1986 moves along the inside of shaft 1908 until barbs 1920 reach the slots, and barbs 1920 then project through the slots, locking shaft 1986 to shaft 1908, and preventing either shaft from withdrawing easily from the vessel wall. Optionally, the barbs are first positioned at a different azimuthal position than the slots, and once the barbs have reached the axial position of the slots, shaft 1986 is rotated until the barbs go through the slots. Alternatively, instead of short slots in shaft 1908, there are long slits extending along shaft 1908. After shaft 1908, including the ends of the slits, has passed through the vessel wall, the barbs, which may be flexible or rigid, project through the slits as shaft 1986 advances through shaft 1908, until the barbs reach the inside of the blood vessel. (f the barbs are rigid, then optionally the barbs push the sides of the opening out of the way as they pass through the wall, or they dilate the tissue, or they cut their way through the wall, similar to the situation described previously where shaft 1986 is outside shaft 1908.)

Alternatively, neither shaft 1986 nor shaft 1908 is inside the other, but the two shafts are side by side. For example, each has the cross-section of half a circle.

It should be understood that, where shaft 1986 is described herein as moving or rotating relative to shaft 1908, optionally shaft 1986 moves or rotates while shaft 1908 stays still, or shaft 1908 moves or rotates while shaft 1986 stays still, or both shaft 1986 and shaft 1908 move or rotate but at different rates. Similar options exist in any other case where one part of the hole former moves relative to another part.

Optionally, once barbs 1920 have passed through the wall, shaft 1986 (or whatever shaft barbs 1920 are attached to) is pulled back until the barbs touch the wall inside the blood vessel. Optionally, this touching is detected by the surgeon because shaft 1986 resists being pulled back further. By putting base 1902 against the outside of the blood vessel wall at the same time as barbs 1920 are touching the inside of the wall, and measuring the relative position of shaft 1986 and base 1902, the surgeon optionally determines the thickness of the wall. If the wall is too compressible, then barbs 1920 may compress the wall significantly before the surgeon feels the resistance of shaft 1986 to being pulled back, and this will affect the accuracy of the thickness measurement. Alternatively, the degree of compression may be measured by comprising the measured thickness at two different controlled retraction forces, for example using one or more springs. The display may be, for example electronic, for example using a linear encoder. Alternatively, the display is mechanical, for example as described below.

Alternatively or additionally, shaft 1986 is advanced until it touches the far wall of the blood vessel, and the relative position of shaft 1986 and base 1902 is used to determine the inner diameter of the blood vessel plus the thickness of the wall. The thickness of the wall may then be inferred by subtracting this distance from the externally measured outer diameter of the blood vessel. Optionally, shaft 1986 is not advanced to the far wall of the blood vessel unless shaft 1908, or at least penetration tip 1904, is covered by shaft 1986, so that the far wall will not be damaged. If penetration tip 1904 is retracted into shaft 1908, then shaft 1908 is optionally used, instead of shaft 1986, to measure the distance to the far wall.

Optionally, whether shaft 1986 is touching the far wall or the inside of the near wall, the relative position of shaft 1986 and base 1902 is measured by markings on a knob or another mechanism on the handle of the hole former, which controls the motion of shaft 1986 relative to base 1902. Additionally or alternatively, a window in the handle allows the surgeon to see an extension of shaft 1986 inside the handle, with markings on shaft 1986 or on the handle to show their relative position. Other mechanisms to measure the relative position of shaft 1986 and base 1902 will be apparent to persons skilled in the art.

Optionally, in addition to or instead of measuring the thickness of the wall, shaft 1986 is used to measure the compressibility of the wall, by measuring the resistance of shaft 1986 to being pulled back further, while base 1902 remains stationary against the outside of the blood vessel. Knowing the thickness and compressibility of the wall may be useful in deciding the best way to cut the wall, or in deciding whether the hole should be cut in this blood vessel at all.

Once shaft 1986 and barbs 1920 have passed through the wall, a cutting lip 1910 on base 1902 starts to cut through the wall to form a hole. Optionally, base 1902 rotates as S cutting lip 1910 cuts through the wall, shown in FIG. 19G. Optionally, the rotation is in one direction. Alternatively, the rotation is back and forth. Optionally, barbs 1920 are brought up to the inner surface of the wall and remain there during the cutting, in order to provide a surface for base 1902 to push against, so base 1902 will not push the whole blood vessel or stretch it during the cutting. Barbs 1920 also prevent the plug from falling into the blood vessel, and allow shaft 1986 to pull the plug out of the wall when shaft 1986 is retracted. Alternatively, instead of cutting lip 1910 cutting a hole through the wall from the outside, the barbs cut or punch a hole through the wall from the inside, or the barbs and cutting lip each cut part way through the wall and meet in the middle of the wall. Optionally, shaft 1986 and/or shaft 1908 do not rotate with base 1902.

Optionally, shaft 1908 is rigidly attached to base 1902, so that shaft 1908 moves together with base 1902 as cutting lip 1910 cuts through the vessel wall, as shown in FIG. 19G. Alternatively, shaft 1908 is attached to base 1902 in such a way that shaft 1908 cannot move axially with respect to base 1902, but shaft 1908 is still free to rotate azimuthally with respect to base 1902. In either case, shaft 1986 is optionally made long enough so that, even when cutting lip 1910 has completely cut through the wall and shaft 1986 has retracted enough so that barbs 1920 are in contact with the wall, penetration tip 1904, which is sharp and can accidentally damage the vessel wall on the other side, is still inside shaft 1986. Designing shaft 1986 to be long enough to do this may depend on estimating a thickness of the blood vessel wall. Alternatively, shaft 1908 is moveable independently of base 1902, and cutting lip 1910 can cut through the wall without shaft 1908 also moving. A potential advantage of this arrangement is that penetration tip 1904 can be kept inside shaft 1986 as base 1902 moves through the wall and while barbs 1920 are against the inside of the wall, even if base 1902 moves further than intended, for example if the wall is thicker than expected. Optionally, one or more controls on the handle of the hole former can lock shaft 1908 to base 1902, or lock shaft 1908 to shaft 1986. Optionally, a device in the handle can cause penetration tip 1904 to retract into shaft 1986 automatically once shaft 1986 has gone through the wall. Optionally, the device can be activated or turned off at the discretion of the surgeon.

Once the vessel wall is cut all the way through, outer shaft 1986, with or without inner shaft 1908, is brought back, taking a plug of cut tissue with it. Optionally, outer shaft 1986 is spring loaded, and automatically goes back as soon as the tissue plug is separated completely from the wall. Base 1902 optionally is left in place temporarily, to keep the blood vessel from leaking.

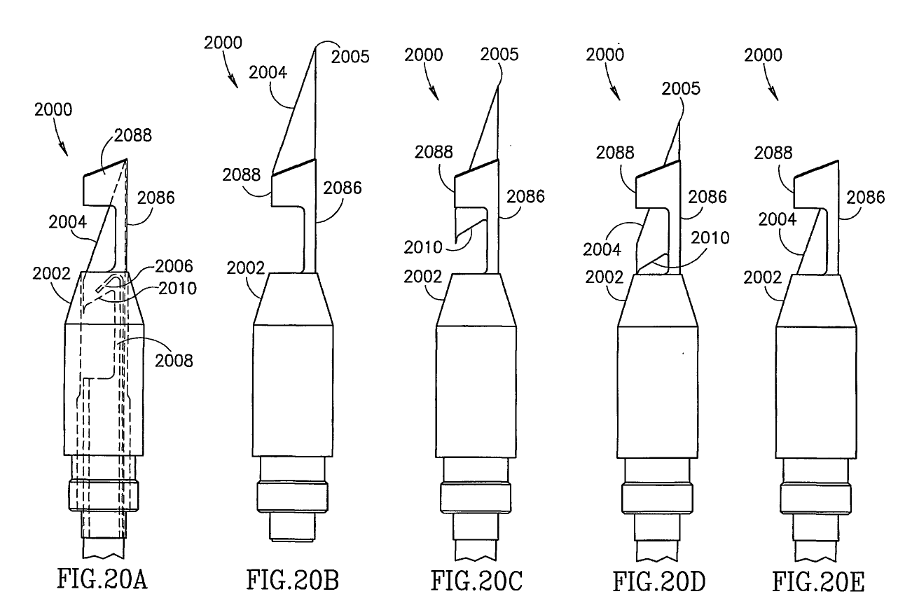

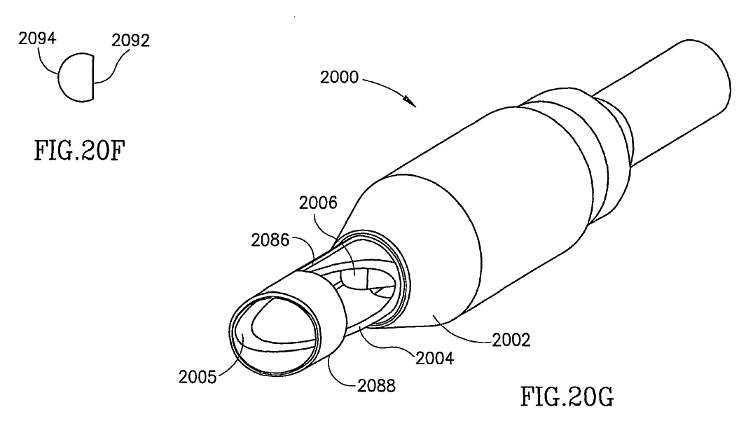

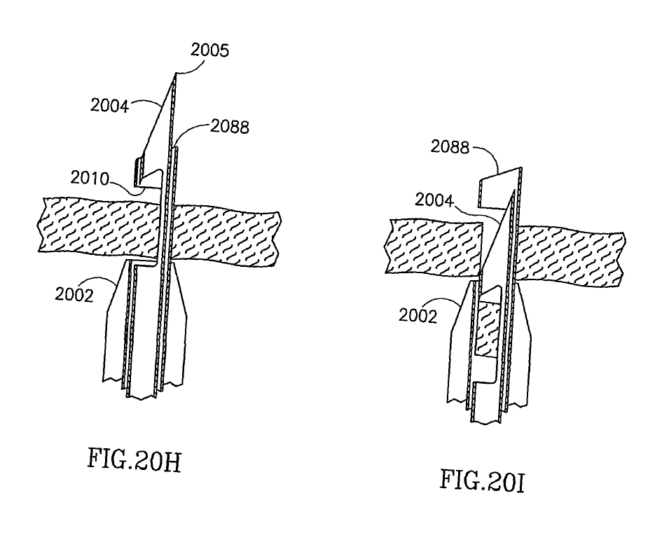

FIG. 20A shows a hole former 2000 according to another exemplary embodiment of the invention. FIG. 20A is a cut-away side view. A perspective view of the same hole former is shown in FIG. 20G. In this embodiment of the invention, there is a penetration head 2004 that is non-axisymmetric. There is a sharp penetration tip 2005 at the end of penetration head 2004. In many cases, a penetration tip of the shape shown in FIG. 20G will make a relatively straight slit in the wall, and the slit will not tend to get longer in an uncontrollable way after it is made. Also, in some cases, the slit will not have a tendency to tear, and these desirable properties will not be sensitive to its orientation relative to the axis of the blood vessel. Optionally, a different shape is used for the penetration tip, and the properties of a slit made by a differently shaped tip may depend on the orientation of the slit, and on the properties of the blood vessel wall. Differently shaped penetration tips may be desirable for different types of blood vessels. Other possible shapes include a tip with a sharp edge that is V-shaped rather than elliptical, a conical tip, and a stiletto-shaped tip.

The whole penetration head enters the blood vessel through the opening made by the penetration tip, and a cutting edge (for cutting the wall from the inside) and/or a wall holder also enter the blood vessel, either as part of the penetration head or together with it. In the embodiment of the invention shown in FIG. 20A, once the penetration head has entered the wall of the blood vessel through this slit-like opening, it is brought back, and a cutting lip 2010 on the back of the penetration head makes an arc-shaped cut in the vessel wall from the inside, next to the slit. Alternatively, instead of cutting lip 2010, there is a blade projecting from the back of penetration head 2004, to make a scissors cut. This arc-shaped cut together with the slit makes a D-shaped hole in the wall.

Optionally, penetration head 2004 is hollow, and there is a hook 2006, or more than one hook, attached to its inner surface which keeps the plug of tissue from slipping off the penetration head into the blood vessel when the penetration head is withdrawn from the blood vessel. Alternatively, the hook is not attached to penetration head 2004, but is attached to the end of its own shaft, which enters the blood vessel through the opening made by penetration tip 2005, at the same time as penetration head 2004 enters the blood vessel, or afterwards.

Making penetration head 2004 hollow also allows cutting lip 2010 to cut through the vessel wall without compressing the tissue. If instead of the cutting lip, there is a blade extending far enough from the back of penetration head 2004, then penetration head 2004 can be solid without compressing the tissue when the blade cuts through the wall. An inner penetration shaft 2008 is attached to the back of penetration head 2004 on one side, the same side that penetration tip 2005 is on, and cutting lip 2010 is also on the back of penetration head 2004, but not at the place where penetration head 2004 is attached to inner shaft 2008. An outer penetration shaft 2086 is shown directly behind inner shaft 2008 (to the right of inner shaft 2008 in FIG. 20A). Optionally, outer shaft 2086 is to the side of inner shaft 2008 instead of behind it (in front or back of inner shaft 2008 in FIG. 20A). An anchor 2088 is optionally attached to the end of outer shaft 2086. All of these parts are mounted in a base 2002. Optionally, anchor 2088 and shaft 2086 move relative to base 2002. Alternatively, anchor 2088 and shaft 2086 are attached rigidly to base 2002.

Optionally, if anchor 2088 and shaft 2086 move relative to base 2002, the relative position of base 2002 and shaft 2086 is used to measure the thickness of the blood vessel wall, as described above for base 1902 and shaft 1986 in FIG. 19. Alternatively or additionally, the compressibility of the wall is also measured, as described above for FIG. 19. Optionally, base 2002 and anchor 2088 hold onto the blood vessel wall after the plug of tissue has been removed, and there is a valve inside base 2002, not shown in the drawings, which keeps blood from leaking out of the blood vessel until another blood vessel can be attached to the hole.

FIGS. 20B-20E illustrates in four steps how hole former 2000 forms a hole in a blood vessel. FIG. 20H shows a side cross-sectional view of the step shown in FIG. 20B, and FIG. 20I shows a side cross-sectional view of the step shown in FIG. 20E. In FIG. 20B, anchor 2088 is set at a position so that the distance between its lower surface 2090 and the top of base 2002 is somewhat more than the expected thickness of the blood vessel wall. Penetration head 2004, extending out beyond anchor 2088, then penetrates the wall of the blood vessel, starting with sharp tip 2005. The tip makes a slit 2092 (shown in FIG. 20F) in the blood vessel, and the slit opens enough for the entire penetration head 2004, and anchor 2088, to enter the blood vessel. The length of the slit and whether it is subject to tearing may depend on the angle at which the tip is oriented with respect to the axis of the blood vessel, although the shape of penetration tip shown in FIG. 20G is designed to work well independent of its orientation. Optionally, hole former 2000 has markings on it, for example further back on the base, to assist the surgeon in orienting penetration head 2004 so that the slit is oriented in a desired direction. Optionally, similar markings are used to help orient asymmetric penetration heads or asymmetric penetration tips in other embodiments of the invention, such as those shown in FIGS. 7A-7E, and 7I.

Once penetration head 2004 and anchor 2088 are inside the blood vessel, the slit partially closes, since it is held open only by shafts 2008 and 2086. Base 2002 optionally is brought up against the outside of the blood vessel, and anchor 2088 optionally is brought back until it is touching the inner surface of the blood vessel. If hole former 2000 is always used for blood vessels with walls of the same thickness, then anchor 2088 optionally is attached at a fixed distance from base 2002.

In FIG. 20C, penetration head 2004 is brought back toward base 2002, and cutting lip 2010, which optionally is shaped like an arc of a circle (missing the part of the circle where penetration head 2004 is attached to shaft 2008) makes a C-shaped cut 2094 (shown in FIG. 20F) in the vessel wall from the inside, with both ends of the C reaching the slit that was made by penetration tip 2005 going into the vessel. Thus, cutting lip 2010 cuts out a hole in the vessel wall. FIG. 20F shows the cut-out hole viewed axially, including slit 2092 and C-shaped cut 2094. Alternatively, cutting lip 2010 could be a different shape. But if cutting lip 2010 is shaped like an arc of a circle, then it can be rotated back and forth as it cuts, while keeping the cut narrow and undistorted. Optionally, the center part 2090 of cutting lip 2010, seen in profile in FIG. 20C, projects out from the rest of cutting lip 2010. Optionally, center part 2090 has a sharp point; alternatively is it is rounded. Center part 2090 allows cutting lip 2010 to make a clean initial cut into the wall, with minimal or no tearing. The cut is then extended by the rest of cutting lip 2010 into a C-shaped cut which reaches the slit. By starting the cut in the middle, away from the slit, cutting lip 2010 does not pull on and distort the slit as it is cutting, or at least does not distort the slit in a lopsided way.

Optionally, hook 2006 engages the back of the plug of tissue that is removed to make the hole, and keeps it from going into the blood vessel when penetration head 2004 is withdrawn from the blood vessel in FIGS. 20D and 20E. Optionally, there is a sharp spike at the end of hook 2006 which penetrates into the plug of tissue, to prevent it from slipping off the one side. Anchor 2088, pressed against the inside of the blood vessel slightly beyond the boundary of the hole, keeps base 2002 pressed against the outside of the blood vessel, so that blood does not leak out. Optionally, penetration head 2004 and the plug of tissue are withdrawn while base 2002 and anchor 2088 remain in place, until the surgeon is ready to attach another blood vessel to the hole. Alternatively, penetration head 2004 and the plug of tissue remain in place together with base 2002 and anchor 2088, and the plug of tissue further reduces leakage of blood, until the surgeon is ready to attach another blood vessel.

Alternatively, there is no hook 2006, and other means are used to keep the plug of tissue from falling into the blood vessel. For example, flexible barbs are located on the inside of anchor 2088. These barbs are pushed downward against the inside surface of anchor 2088 by penetration head 2004, until penetration head 2004 is pulled out of the blood vessel, leaving the plug of tissue in place but separated from the blood vessel wall, and largely plugging the hole and keeping blood from leaking out. When anchor 2088 is withdrawn from the blood vessel, then the barbs engage the plug and pull it out also.

Alternatively, the cutting is provided by base 2002 or by an additional outer cutting tube. Alternatively or additionally, scissors type cutting or anvil type cutting is provided, for example, between lip 2010 and base 2088.

In FIGS. 20A-20E and 20G, anchor 2088 is concentric with penetration head 2004 and surrounds it. Alternatively, anchor 2088 does not go all the way around penetration head 2004, but only goes part way around it, for example, head 2004 traveling in a groove formed in anchor 2088 or along side it.

In an exemplary embodiment of the invention, head 2004 defines a cutting surface on its proximal side that corresponds to the sections of anchor 2088 that are not transaxially slotted. This may be provided, for example, by head 2004 traveling in a groove in anchor 2088 and wings extending from head 2004, out of the groove and covering the unslotted parts, so that head 2004 partly encloses anchor 2088 and anchor 2088 partly encloses head 2004.

It should be noted that while some of the above embodiments have been described with head 2004 mounted on a shaft that is outside anchor 2088, in some embodiments, head 2004 is mounted on a shaft that is at least partly surrounded by anchor 2088, albeit not in a coaxial configuration with relation to anchor 2088. Alternatively or additionally, head 2004 is symmetrically designed and arranged relative to anchor 2088.

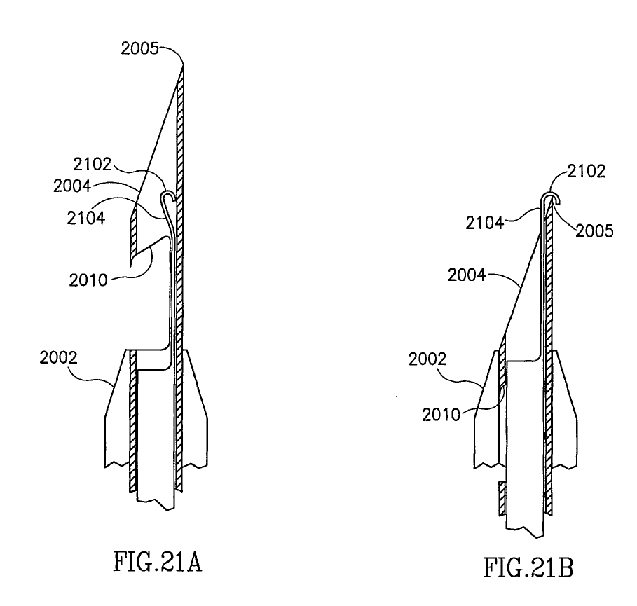

FIGS. 21A and 21B show a hole former according to another exemplary embodiment of the invention. The hole cutter shown in FIGS. 21A and 21B is similar to that shown in FIGS. 20A-201, but it also includes a penetration head catch 2102. Optionally, penetration head catch 2102 is fixed to base 2002, as shown in FIGS. 21A and 21B. Alternatively, penetration head catch 2102 is fixed to shaft 2086, if shaft 2086 exists, or penetration head catch 2102 is an independent part of the hole former. When penetration head 2004 is some distance away from base 2002, as it is when penetration head 2004 has finished penetrating the blood vessel wall, penetration head catch 2102 is located inside the hollow penetration head, as shown in FIG. 21A. A spring 2104 keeps penetration head catch 2102 pressed against the inside surface of penetration head 2004. As penetration head 2004 is retracted, as shown in FIG. 21B, catch 2102 extends past the tip of penetration head 2004, and spring 2104 pushes catch 2102 over the tip. If catch 2102 is fixed to base 2002, this locks penetration head 2004 to base 2002, and penetration head 2004 is prevented from accidentally moving away from base 2002, where it may damage the blood vessel wall on the other side of the blood vessel, for example. Similarly, penetration head 2004 is constrained from moving if catch 2102 is fixed to another part of the hole former, for example a shaft similar to shaft 2086 in FIG. 20. Whether or not penetration head 2004 is constrained from moving by catch 2012, catch 2102 optionally covers a sharp tip on penetration head 2004, and may prevent it from damaging the blood vessel wall. Optionally, catch 2102 is not fixed to base 2002 or to any other part of the hole former, and does not automatically slip over penetration head 2004 when penetration head 2004 is retracted, but catch 2102 is manipulated to slip over penetration head 2004 by the surgeon, at any desired time. Optionally, there is a control in the handle of the hole former enabling the surgeon to manipulate catch 2102. Additionally or alternatively, there is a control in the handle of the hole former which allows the surgeon to release catch 2102, for example if it was set by mistake.

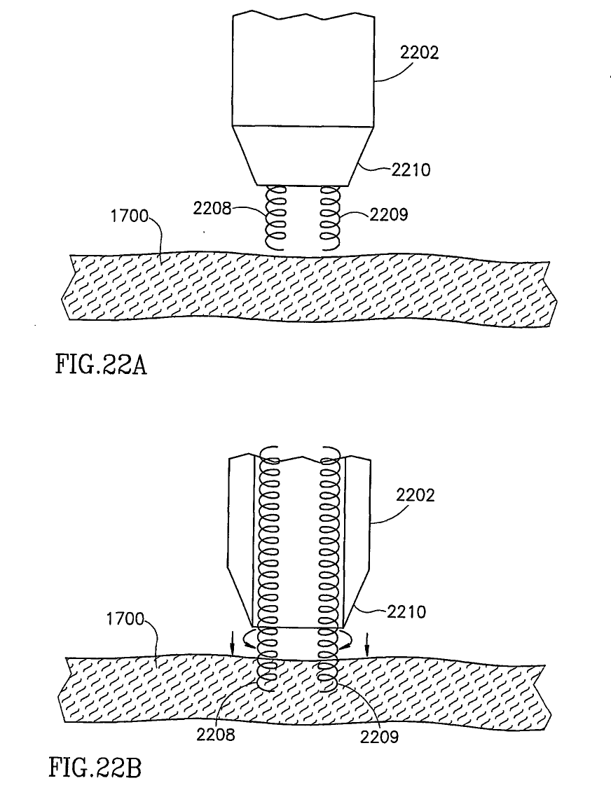

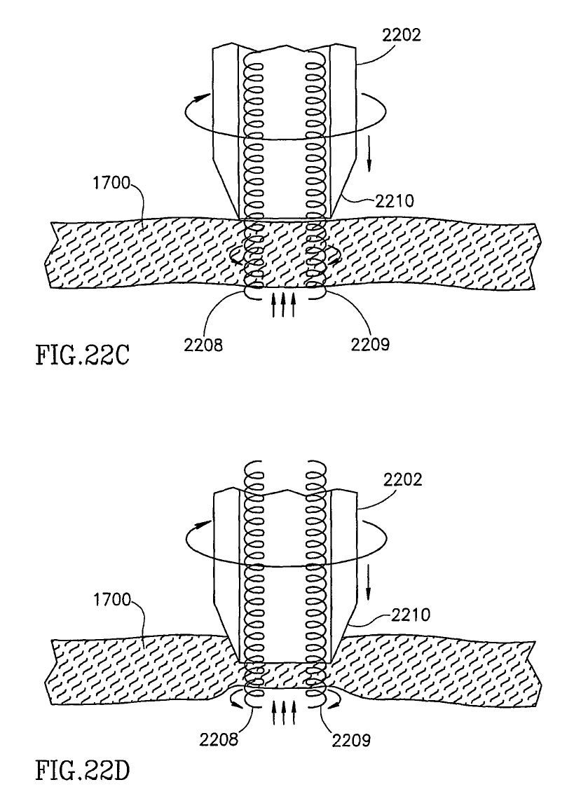

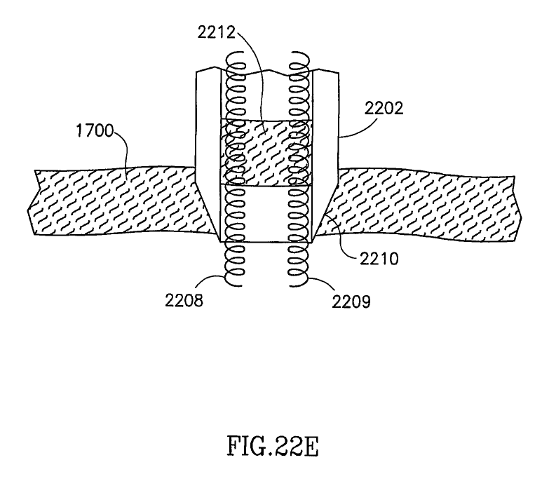

FIGS. 22A-22E show a hole former with two helical penetration shafts 2208 and 2209, attached to a base 2202 with a cutting surface 2210. The five drawings show the procedure by which the hole former makes a hole in a blood vessel wall 1700. In FIG. 22A, helical shafts 2208 and 2209, with opposite helicities, are located just outside wall 1700. In FIG. 22B, the two shafts rotate in opposite directions as they penetrate the wall, creating two helical channels matching the helicities of the shafts. In FIG. 22C, the shafts have penetrated the wall, and cutting edge 2210 starts to cut a circular hole in the wall, optionally rotating as it cuts. Alternatively cutting edge 2210 does not rotate, but cuts the wall with a punching motion. In FIG. 22D, shafts 2208 and 2209 retract with respect to cutting edge 2210 and base 2202, without rotating, as cutting edge 2202 cuts through the wall, providing cutting edge 2210 with something to push against as it cuts. In FIG. 22E, the cutting edge 2210 has completely cut through the wall, leaving a cut out plug of tissue 2212. Optionally, shafts 2208 and 2209 then retract further into base 2202 without rotating, pulling out tissue plug 2212. Alternatively, shafts 2208 and 2209 rotate in the same direction as they did while penetrating the wall, while not retracting with respect to base 2202, which also causes plug 2212 to be pulled into base 2202, or shafts 2208 and 2209 rotate and retract at the same time.

Optionally, instead of two shafts 2208 and 2209 with opposite helicities, there is only one shaft. A potential advantage of having two shafts with opposite helicities is that the torques exerted by the two shafts may cancel out, so that they do not exert any net torque on the blood vessel. If there are two shafts, they may be side by side, as shown in FIGS. 22A-22E, or they may be overlapping, or even coaxial. If the two shafts are coaxial, they can avoid intersecting each other by having at least slightly different diameters. Another potential advantage of having two shafts of opposite helicities, as opposed to a single helical shaft with its axis coinciding with the axis of circular tissue plug 2212, is that tissue plug 2212 cannot slip off the shafts by twisting, as the shafts rotate and/or retract. This advantage could also apply to two shafts with the same helicity, if they are not coaxial, or even to a single helical shaft that is not coaxial with the circular tissue plug. In order for the tissue plug to slip off the shaft or shafts in those cases, the tissue plug would have to be stretched, compressed, and/or sheared, rather than simply twisting rigidly as in the case of a single centered helical shaft.

The hole former shown in FIGS. 22A-22E has the potential advantage that the penetration of the wall can be reversed at any time simply by rotating the shafts in the opposite direction that they were rotated in while penetrating the wall, while retracting them. If this is done, the shafts will only leave small openings in the wall, which may close up by themselves with minimal loss of blood. This can be done even after cutting surface 2210 has started to cut through the wall. The penetration shafts could be used to measure the thickness and/or compressibility of the blood vessel wall, for example, using a method similar to that described above for FIGS. 19 and 20. If the wall does not have a thickness and/or compressibility within the expected range, and the surgeon decides that the blood vessel is therefore not suitable for the procedure being performed, then the penetration shafts can be removed with little damage, and another blood vessel can be chosen.

The above description has focused on devices that are applied from outside a blood vessel. However, they can also be applied from inside of blood vessels.

In an exemplary embodiment of the invention, the design is optionally changed to accommodate one or more of the following factors:
(a) which layer of the blood vessel is to be cut more precisely;
(b) what type of cutting action to apply to each blood vessel layer;
(c) disposal of the tissue plug (if any) to outside the blood vessel or to inside the delivery system; and
(d) desired cut profile.

In one example of an inside-out punch, the tissue receptacle is located on the base and has a cutting lip that extends forward. In another example, the tissue receptacle is on the penetration head but the base advances forwards towards the receptacle.

In addition, the aperture forming systems may be provided in several sizes, for example, two, three or more sizes.

It should be noted that the elements described as tubes are not generally required to be tubes. In one example, the apertured base tube can be replaced by a slotted solid rod, in which the slot carries a shaft for retraction of the penetration head. The shaft need not attach to the center of the penetration head.

It should also be noted that hole formers can be used to create incomplete removal of plugs, for example, to create rectangular or triangular flaps.

In an exemplary embodiment of the invention, the above devices are used in combination with anastomosis-related tools as described in PCT applications and publications WO 99/62415, WO 00/56226, WO 00/56228, WO 01/41623, WO 01/41624, PCT/IL01/00267, PCT/IL01/00069, PCT/IL01/00074, and PCT/IL01/00266, the disclosures of which are incorporated herein by reference. However, they may also be used as stand alone devices or as part of surgical kits for other uses and/or anastomosis connectors.

It will be appreciated that the above described methods and devices of vascular manipulation may be varied in many ways, including, changing the order of steps, the exact materials used for the devices, which vessel is a “side” side, which vessel (or graft) is an “end” side of an end-to-side anastomosis and whether the device is used from inside or from outside a blood vessel. Further, in the mechanical embodiments, the location of various elements may be switched, without exceeding the spirit of the disclosure, for example, switching the moving elements for non-moving elements where relative motion is required. In addition, a multiplicity of various features, both of methods and of devices have been described. It should be appreciated that different features may be combined in different ways. In particular, not all the features shown above in a particular embodiment are necessary in every similar exemplary embodiment of the invention. Further, combinations of the above features, from different described embodiments are also considered to be within the scope of some exemplary embodiments of the invention. In addition, some of the features of the invention described herein may be adapted for use with prior art devices, in accordance with other exemplary embodiments of the invention. The particular geometric forms used to illustrate the invention should not be considered as necessarily limiting the invention in its broadest aspect to only those forms, for example, where a circular lumen is shown, in other embodiments an oval lumen may be used.

Also within the scope of the invention are surgical kits which include sets of medical devices suitable for making a single or a small number of anastomosis connections and/or apertures. Measurements are provided to serve only as exemplary measurements for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. Hole forming apparatus for forming a hole in a blood vessel, comprising:
a penetration shaft, having a tip adapted to be inserted through a wall of a blood vessel;
a tissue holder, configured to hold a portion of said wall, said holder being activated to perform said holding separately from an insertion of said penetration shaft through said wall; and
a cutting surface adapted to cut through the wall.

2. Hole forming apparatus according to claim 1, wherein said tissue holder comprises a rigid barb.

3. Hole forming apparatus according to claim 1, wherein said tissue holder comprises a flexible barb.

4. Hole forming apparatus according to claim 1, wherein said tissue holder comprises a disk.

5. Hole forming apparatus according to claim 1, wherein the tissue holder comprises a hollow tube surrounding the penetration shaft.

6. Apparatus according to claim 5, wherein said hollow tube is configured to be advanced along said penetration shaft such that said tip is wholly contained by said hollow tube.

7. Apparatus according to claim 6, wherein said hollow tube is long enough to contain said tip at least until said wall is cut through by said cutting surface.

8. Hole forming apparatus according to claim 1, wherein the penetration shaft at least partially encloses said tissue holder.

9. Hole forming apparatus according to claim 8, wherein said tissue holder comprises at least one flexible tissue holding element and wherein the penetration shaft comprises at least one slot wide enough to receive said flexible element therethrough and wherein said tissue holder and said penetration shaft are configured for selectively positioning said penetration shaft relative to said tissue holder such that said tissue holding element and said slot align.

10. Hole forming apparatus according to claim 9, wherein said tissue holder and said penetration shaft are configured such that said tissue holding element is axially displaced from said slot.

11. Hole forming apparatus according to claim 9, wherein said tissue holder and said penetration shaft are configured such that said tissue holding element is angularly displaced from said slot.

12. Hole forming apparatus according to claim 1, wherein said tissue holder lies alongside said penetration shaft.

13. Hole forming apparatus according to claim 1, wherein said tissue holder is mounted on said penetration shaft and comprising a holder releaser configured to selectively release said holder to hold tissue.

14. Hole forming apparatus according to claim 13, wherein said holder releaser comprises a hollow shaft which at least partially encloses said penetration shaft.

15. Hole forming apparatus according to claim 13, wherein the holder releaser lies alongside said penetration shaft.

16. Hole forming apparatus according to claim 13, wherein said holder releaser covers said tissue holder during said insertion of said penetration shaft into said wall.

17. Hole forming apparatus according to claim 16, wherein said holder releaser resiliently compresses said tissue holder during said insertion of said penetration shaft into said wall.

18. Hole forming apparatus according to claim 1, wherein said tissue holder is configured to be advanced along said penetration shaft after said insertion.

19. Apparatus according to claim 1, wherein said tissue holder is configured to be axially moved relative to said penetration shaft, thereby activating said tissue holder.

20. Apparatus according to claim 1, wherein said tissue holder is configured to be rotated relative to said penetration shaft, thereby activating said tissue holder.

21. Apparatus according to claim 1, wherein said tissue holder is configured to retract relative to said cutting surface during said hole forming.

22. Apparatus according to claim 21, wherein said tissue holder is spring loaded to retract.

23. Apparatus according to claim 21, wherein said retraction is mechanically coupled to a rotation of said cutting surface.

24. Apparatus according to claim I, wherein said penetration shaft has a fixed axial position relative to said cutting surface at least after said insertion.

25. Apparatus according to claim 1, wherein said penetration shaft is axially retractable relative to said cutting surface.

26. Apparatus according to claim 1, wherein said cutting surface is confirmed to cut by rotation,

27. Apparatus according to claim 26, wherein said cutting surface is not rotationally fixed to said tissue holder.

28. Apparatus according to claim 1, wherein said cutting surface is configured to cut by from an opposite side of said wall from said penetration tip.

29. Apparatus according to claim 1, wherein said cutting surface is configured to cut by from a same side of said wall from said penetration tip.

30. Apparatus according to claim 1, wherein said penetration tip is configured to enter said wall from an outside of said vessel.

31. Apparatus according to claim 1, wherein said tissue holder engages a wall of said vessel.

32. Apparatus according to claim 1, wherein said tissue holder contacts a wall of said vessel at a stop location and thereby prevents relative motion of said wall in a direction of said stop location.

33. Hole forming apparatus for forming an opening in a blood vessel, comprising:
a penetration shaft, having a tip adapted to be inserted through a wall of a blood vessel;
a cutting surface adapted to cut through the wall and having a fixed axial position relative to said penetration shaft tip; and
a tip protector, axially movable to protect the tip of the penetration shaft from damaging the blood vessel after the penetration shaft is inserted through the wall of the blood vessel.

34. Hole forming apparatus according to claim 33, wherein said tip protector comprises at least one tissue holding element configured to hold at least a portion of said wall after said insertion.

35. Apparatus according to claim 34, wherein said tip protector has a length, distal of said tissue holder, greater than an axial distance between said tip and said cutting surface.

36. Apparatus according to claim 33, wherein the tip protector comprises a hollow tube which surrounds the penetration shaft.

37. Hole forming apparatus for forming a hole in a blood vessel, comprising:
a slit-forming penetration head having a tip on a distal end thereof and configured to be inserted into, and form a slit, in a blood vessel wall; and
a cutting surface not-contiguous with said tip, configured to cut said wall such that said cut and said slit link to provide a boundary cut of said wall, defining said hole.

38. Apparatus according to claim 37, wherein said cutting surface is formed on a proximal side of said head.

39. Apparatus according to claim 37, wherein said tip is asymmetrically located on said head relative to an axis of said apparatus.

40. Hole forming apparatus according to claim 37, wherein the penetration head is solid.

41. Hole forming apparatus according to claim 37, wherein the penetration head is hollow.

42. Apparatus according to claim 41, and including at least one head tissue holder positioned inside said head and configured to prevent a cut out portion of the wall of the blood vessel from passing through said head in a direction of said tip.

43. Apparatus according to claim 42, wherein said tissue holder is fixed to said head.

44. Apparatus according to claim 42, wherein said tissue holder comprises a hook.

45. Apparatus according to claim 42, wherein said penetration head has an arcuate profile when viewed along its axis.

46. Hole forming apparatus according to claim 37, and including a sharpened tip at a proximal end of the penetration head.

47. Hole forming apparatus according to claim 46, wherein said sharpened tip is on a same plane as said penetration tip and an axis of said penetration head.

48. Hole forming apparatus according to claim 37, and including an anchor comprising a member defining a trans-axial slot, said slot having a width sufficient to receive a thickness of said wall.

49. Hole forming apparatus according to claim 48, wherein said member comprises a tube defining a lumen having a diameter sufficient to enclose said penetration head.

50. Hole forming apparatus according to claim 48, wherein said member only partly surrounds said penetration head.

51. Hole forming apparatus according to claim 48 and including an anchor tissue holder attached to said anchor, which anchor tissue holder prevents a cut out portion of the wall of the blood vessel from passing into the blood vessel.

52. Hole forming apparatus according to claim 48, wherein said member has a distal end.

53. Apparatus according to claim 52, wherein said distal end comprises an anchor cutting surface.

54. Apparatus according to claim 52, wherein said distal end comprises said cutting surface.

55. Apparatus according to claim 52, wherein said distal end and a proximal end of said penetration head cooperate to provide a scissors cutting action.

56. Apparatus according to claim 52, wherein said distal end and a proximal end of said penetration head cooperate to provide an anvil cutting action.

57. Hole forming apparatus for forming a hole in a blood vessel, comprising:
a penetration head adapted to penetrate a blood vessel wall;
a slotted anchor, defining a trans-axial slot having a width sufficient to receive a thickness of said wall; and
a cutting surface configured to cut said wall while said wall is held by said slotted anchor.

58. Apparatus according to claim 57, wherein said anchor comprises a trans-axially-slotted tube.

59. Hole forming apparatus according to claim 57, wherein the penetration head comprises a distal portion of the anchor.

60. Hole forming apparatus according to claim 57, and including a tissue holder which prevents a cut out portion of the wall of the blood vessel from passing into the blood vessel.

61. Apparatus according to claim 57, wherein said cutting surface is formed on a proximal side of said penetration head.

62. Hole forming apparatus for forming a hole in a blood vessel, comprising:
a penetration head having a tip adapted to penetrate a blood vessel wall and defining an axial lumen; and
a tip protector configured to pass through said lumen and protect said tip from damaging the blood vessel after said penetration.

63. Apparatus according to claim 62, wherein said tip protector comprises a folded tab defining a receptacle configured to receive said tip therein.

64. Apparatus according to claim 63, wherein said tab is resiliently distorted during passage through said lumen.

65. Apparatus according to claim 62, wherein said tip protector has a fixed axial location relative to said apparatus such that retraction of said penetration head causes said tip protector to pass through said lumen and protect said tip.

66. Apparatus according to claim 62, wherein said tip protector comprises a control for manually positioning said tip protector to selectively protect said tip.

67. Hole forming apparatus for forming a hole in a blood vessel, comprising:
a tissue holder configured to be inserted through a blood vessel wall;
a base configured to be positioning on an opposite side of said wall; and
a display coupled to said tissue holder and said base and configured to show an indication based on a relative distance between said tissue holder and said base.

68. Apparatus according to claim 67, wherein said tissue holder includes at least one tissue retraction prevention element which prevents retraction of said tissue holder back through said wall.

69. Apparatus according to claim 67, wherein said display is mechanically coupled to said holder and said base.

70. Apparatus according to claim 67, wherein said base contacts said wall with a blood vessel wall cutting surface.

71. Apparatus according to claim 67, comprising a spring which retracts said holder relative to said base.

72. Apparatus according to claim 67, wherein said display converts said distance into a measure of compressibility of said wall.

73. Apparatus according to claim 67, wherein said display converts said distance into a measure of a thickness of said wall.

74. Hole forming apparatus for forming a hole in a blood vessel, comprising:
a penetration head mounted on a shaft and adapted to penetrate a blood vessel wall; and
a tissue receptacle configured to receive said wall after said penetration,
wherein said shaft is not co-axial with said tissue receptacle.

75. Apparatus according to claim 74, wherein said shaft is outside of said tissue receptacle.

76. Hole forming apparatus for forming a hole in a blood vessel, comprising:
a plurality of helical coils adapted to penetrate and engage a blood vessel wall; and
a base defining a cutting surface for cutting said wall and configured to be axially moved relative to said helical coils.

77. Apparatus according to claim 76, wherein said coils include at least two coils having opposite helicity.

78. Apparatus according to claim 76, wherein said coils include at least two coaxial coils.

79. Apparatus according to claim 76, wherein said coils include at least two non-coaxial coils.

80. A method of forming a hole in the wall of a vessel, comprising:
providing a hole former comprising a shaft having a penetrating tip formed at a distal end of the shaft, an outer shaft disposed about the shaft and configured to be slideable with respect to the shaft, the outer shaft having at least one projecting element, and a base disposed about the outer shaft having a cutting lip, the base configured to be slideable relative to the outer shaft,
penetrating a vessel wall with the penetrating tip to form an opening;
passing the outer shaft and the at least one projecting element through the opening; and
cutting the vessel wall with the cutting lip of the base to form a hole in the wall of the vessel.

81. A method according to claim 80, comprising retracting the outer shaft such that the at least one projecting element contacts the inner surface of the vessel wall prior to said cutting.

82. The method of claim 80, wherein the shaft and the base are fixed relative to one another.

83. The method of claim 80, wherein the shaft is moveable independently of the base.

84. The method of claim 83, wherein the penetrating tip is retracted into the outer shaft one the outer shaft has passed through the opening.

85. The method of claim 80, wherein the outer shaft shields the inner surface of the vessel wall from the penetrating tip during the cutting step,

86. The method of claim 80, wherein the base is rotated to out the hole in the vessel wall.
